(19) United States
(12) Patent Application Publication
(10) Pub. No.: US 2025/0307658 A1
(43) Pub. Date: Oct. 2, 2025

(54) LARGE LANGUAGE MODEL AGENT FOR AUTOMATED GENE-EDITING EXPERIMENT DESIGN

(71) Applicants: The Trustees of Princeton University, Princeton, NJ (US); Stanford University, Stanford, CA (US)

(72) Inventors: Mengdi Wang, Princeton, NJ (US); Kaixuan Huang, Princeton, NJ (US); Yuanhao Qu, San Mateo, CA (US); Le Cong, Mountain View, CA (US)

(73) Assignees: The Trustees of Princeton University, Princeton, NJ (US); Stanford University, Stanford, CA (US)

(21) Appl. No.: 19/093,928

(22) Filed: Mar. 28, 2025

Related U.S. Application Data
(60) Provisional application No. 63/571,707, filed on Mar. 29, 2024.

(51) Int. Cl. G06N 3/126; G06F 16/334; G06F 16/338; G16B 40/20

(52) U.S. Cl. CPC G06N 3/126; G06F 16/3344; G06F 16/338; G16B 40/20

(57) ABSTRACT

A platform for automated design of gene-editing experiments includes one or more processing units and a non-transitory computer-readable storage device. The storage device contains instructions that, when executed, configure the processing units to perform a method. The method includes receiving a meta request with information about a requested gene-editing experiment, configuring an ordered list of tasks via a reasoning framework, and implementing tasks via a Task Executor module utilizing state machines. The Task Executor connects to external APIs, provides instructions to a User-Proxy Agent module, and receives user input. The User-Proxy Agent forms prompts based on current state instructions, user requests, interaction history, and API results to determine appropriate actions. The platform outputs recommendations responsive to the meta request.

### CROSS-REFERENCE TO RELATED APPLICATIONS

This application claims priority to U.S. Provisional Patent Application No. 63/571,707, filed Mar. 29, 2024, which is hereby incorporated by reference in its entirety.

### FIELD OF INVENTION

The present disclosure relates to artificial intelligence systems for biological research, and more particularly to a large language model-based agent system for automating the design of CRISPR gene-editing experiments.

### BACKGROUND

Genome engineering technology has transformed biomedical research by enabling precise modifications to genetic information. This field encompasses various techniques for altering DNA sequences within living organisms, with applications ranging from basic scientific research to potential therapeutic interventions. Among these techniques, CRISPR-Cas systems have emerged as a widely adopted tool due to their efficiency and versatility.

CRISPR, which stands for Clustered Regularly Interspaced Short Palindromic Repeats, was originally discovered as part of bacterial immune systems. Researchers later adapted this natural mechanism into a programmable gene-editing tool. The CRISPR-Cas system typically consists of a guide RNA (gRNA) that directs a Cas nuclease to a specific DNA sequence, where it can make targeted modifications.

As the field of genome engineering has advanced, researchers have developed various CRISPR-based techniques beyond simple gene knockout. These include methods for activating or repressing gene expression (CRISPRa/i), introducing precise base changes without double-strand breaks (base editing), and making small insertions or deletions (prime editing). Each of these approaches has its own set of considerations and design parameters.

Designing effective gene-editing experiments requires a deep understanding of both the CRISPR technology and the biological system under investigation. Researchers must consider factors such as the choice of CRISPR system, guide RNA design, delivery method, and potential off-target effects. Additionally, validating the results of gene-editing experiments often involves complex molecular biology techniques and data analysis.

The complexity of gene-editing experimental design can present challenges, particularly for researchers who are new to the field or working with unfamiliar biological systems. There is a general interest in tools and resources that can assist in streamlining the experimental design process, potentially reducing the time and resources required to plan and execute gene-editing studies.

Artificial intelligence and machine learning approaches have shown promise in various areas of biological research. These computational methods can process large amounts of data and potentially identify patterns or make predictions that may not be immediately apparent to human researchers. There is ongoing exploration of how such approaches might be applied to enhance the design and execution of gene-editing experiments.

As the field of genome engineering continues to evolve, there is a general focus on improving the efficiency, specificity, and accessibility of gene-editing techniques. This includes efforts to refine existing tools, develop new methodologies, and create resources that can support researchers in designing and implementing gene-editing experiments across a wide range of applications.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a platform for automated design of gene-editing experiments is provided. The platform includes one or more processing units and a non-transitory computer-readable storage device operably coupled to the one or more processing units. The non-transitory computer-readable storage device contains instructions that, when executed, configure the one or more processing units to, collectively, perform a method. The method includes receiving a meta request, the meta request including information about a requested gene-editing experiment. The method further includes configuring, via a reasoning framework, an ordered list of tasks required to achieve the requested gene-editing experiment based on the information. The reasoning framework is configured to sequentially send each task in the ordered list of tasks, and optionally a previous result, to a Task Executor module and receive a result from the Task Executor Module responsive to the task. The method also includes implementing, via the Task Executor module, a task received from the reasoning framework. The Task Executor utilizes state machines to decompose sub-goals and is configured to connect to one or more external application programming interfaces (APIs) by sending an API call to a Tool Provider module and receiving a result, provide instructions to a User-Proxy Agent module and receive user input responsive to the instructions from the User-Proxy Agent, and send feedback to the User-Proxy Agent based on the task and/or user input. The method further includes forming a prompt, via the User-Proxy Agent module, based on an instruction inherent to a current state from the Task Executor module, a request made by the user, a history of past interactions within the current task session, results from external APIs, or a combination thereof, then using the prompt with the User-Proxy Agent module to determine an appropriate next action. The current state encapsulates a description of a current task and any input required from a user. The platform is configured to output one or more recommendations responsive to the meta request.

According to other aspects of the present disclosure, the platform may include one or more of the following features. The reasoning framework may comprise a large language model configured to decompose the meta request into the ordered list of tasks. The large language model may be trained using a dataset comprising curated question-and-answer pairs derived from gene-editing discussions. The large language model may be fine-tuned using a technique selected from the group consisting of full parameter fine-tuning and quantized low-rank adaptation (QLoRA) fine-tuning.

According to another aspect of the present disclosure, a method for selecting gene editing delivery methods is provided. The method includes extracting parameters from user inputs related to gene editing, performing a literature search based on the extracted parameters, ranking candidate delivery methods using citations from the literature search results, and outputting a ranked list of candidate delivery methods.

According to other aspects of the present disclosure, the method for selecting gene editing delivery methods may include one or more of the following features. The method may further comprise categorizing the user inputs into one of a plurality of predefined biological categories, wherein the literature search is performed based on the categorized biological category. The plurality of predefined biological categories may comprise: mammalian in vivo, mammalian embryos, mammalian primary cells or stem cells ex vivo, mammalian cell lines with strong evidence of high-efficiency transfection, mammalian cell lines or organoids without strong evidence of high-efficiency transfection, human in vivo or human embryos, and bacteria, viruses, and other organisms. Ranking the candidate delivery methods may comprise retrieving a predefined set of delivery methods associated with the categorized biological category, calculating a score for each delivery method based on the number of citations from the literature search results, and ordering the delivery methods based on the calculated scores.

According to another aspect of the present disclosure, a method for training a gene editing model is provided. The method includes obtaining a dataset of gene editing discussions from a public forum, preprocessing the dataset to extract question-answer pairs, fine-tuning a pre-trained language model using the extracted question-answer pairs, and storing the fine-tuned model for subsequent use in gene editing tasks.

According to other aspects of the present disclosure, the method for training a gene editing model may include one or more of the following features. Preprocessing the dataset may comprise anonymizing personal information in the discussions, extracting question-answer pairs from individual discussion threads, and filtering the extracted pairs to remove irrelevant or low-quality content. Fine-tuning the pre-trained language model may comprise using a technique selected from the group consisting of full parameter fine-tuning and quantized low-rank adaptation (QLoRA) fine-tuning. The method may further comprise evaluating the fine-tuned model using a test set of gene editing questions and comparing the performance of the fine-tuned model to the pre-trained model on the test set.

According to another aspect of the present disclosure, a method for gene editing inference is provided. The method includes receiving a gene editing query, processing the query using a model trained with fine-tuning on gene editing discussions, retrieving relevant information from a curated knowledge base of gene editing literature, synthesizing an answer based on the processed query and retrieved information, and outputting the synthesized answer.

According to other aspects of the present disclosure, the method for gene editing inference may include one or more of the following features. Retrieving relevant information from the curated knowledge base may comprise embedding the gene editing query and documents in the knowledge base into semantic vectors, performing a similarity search to identify the most relevant documents based on cosine similarity between the query vector and document vectors, and summarizing the identified relevant documents in relation to the gene editing query. Synthesizing the answer may comprise combining information from the processed query, the retrieved relevant information, and a response generated by a fine-tuned large language model trained on gene editing discussions, and generating a concise answer that addresses the specific aspects of the gene editing query.

According to another aspect of the present disclosure, a method for designing guide RNA for gene editing is provided. The method includes receiving a user request for guide RNA design, extracting relevant parameters from the user request, accessing a pre-designed guide RNA table, applying a chain-of-table methodology to process the pre-designed guide RNA table based on the extracted parameters, selecting guide RNA sequences from the processed table, and outputting the selected guide RNA sequences.

According to other aspects of the present disclosure, the method for designing guide RNA for gene editing may include one or more of the following features. Applying the chain-of-table methodology may comprise selecting rows from the pre-designed guide RNA table where specified columns match given values, ordering the selected rows based on values in a specified column, and returning a top number of rows from the ordered selection.

According to another aspect of the present disclosure, a system for automated design of gene-editing experiments is provided. The system includes a User-Proxy Agent module configured to interact with a user and process user inputs, a reasoning framework configured to decompose a gene editing request into an ordered list of tasks, a Task Executor module configured to implement tasks using state machines, a Tool Provider module configured to connect to external APIs, and a non-transitory computer-readable storage device containing instructions that, when executed, cause the system to perform a method. The method includes receiving a gene editing request from the user via the User-Proxy Agent module, decomposing the request into tasks using the reasoning framework, sequentially executing the tasks using the Task Executor module, and outputting gene editing experiment design recommendations to the user via the User-Proxy Agent module.

According to other aspects of the present disclosure, the system for automated design of gene-editing experiments may include one or more of the following features. The reasoning framework may comprise a large language model trained on a dataset of curated question-answer pairs derived from gene-editing discussions. The large language model may be fine-tuned using a technique selected from the group consisting of full parameter fine-tuning and quantized low-rank adaptation (QLoRA) fine-tuning.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1A illustrates a block diagram of a system for automated design of gene-editing experiments, according to aspects of the present disclosure.

FIG. 1B illustrates a block diagram of components of CRISPR-GPT according to a first embodiment.

FIG. 1C illustrates a block diagram of components of CRISPR-GPT according to a second embodiment.

FIG. 2 illustrates a diagram showing a task decomposition process and state machine implementation, according to aspects of the present disclosure.

FIG. 3 illustrates a diagram providing an overview of CRISPR-GPT's interactive modules for gene-editing experimental design, with schematics illustrating the functionality of three modules, accompanied by examples of their applications.

FIG. 4 illustrates an example user interface for an embodiment of CRISPR-GPT according to aspects of the present disclosure.

FIGS. 5A-5F illustrate example workflows outlining general tasks involved in gene-editing experimental designs as facilitated by CRISPR-GPT.

FIGS. 6A-6D are graphs showing evaluations results of comparative performance of CRISPR-GPT and ChatGPT 3.5/4.0 in total (6A) and in a range of gene-editing experiment design tasks across three different modes: MetaMode (6B), AutoMode (6C), and QAMode (6D), according to aspects of the present disclosure.

FIGS. 7A-7B provide a flowchart of an example schematic showing the workflow of human-AI interaction for a wet-lab demonstration of collaboration in performing a gene-knockout experiment.

FIG. 8 illustrates a bar graph showing editing efficiency values for different targets from next generation sequencing, according to an embodiment.

FIG. 9A illustrates an example of an LLM Planner Agent automatically breaking down a user's meta-request to a sequence of tasks, then assembling a customized workflow of the chained tasks to meet the user's needs.

FIGS. 9B-9D are graphs showing Auto-mode LLM planner evaluations using a gene-editing planning test set.

FIG. 10 is a table summarizing the number of user interactions and specific inputs required by the LLM User-Proxy Agent to complete the 22 unique experiment design tasks automated by CRISPR-GPT.

FIG. 11 illustrates a design of delivery method selection agent in CRISPR-GPT, showing the workflow, example request, and a series of agent thoughts-actions to identify most suitable delivery methods for the user's needs.

FIG. 12 illustrates a design of guideRNA design agent in CRISPR-GPT, showing the workflow, example request, and a series of agent thoughts-actions to select top-ranked gRNA customized to user's request.

FIG. 13 illustrates a design of QA Mode in CRISPR-GPT, showing the workflow, example request, and a series of agent thoughts-actions to answer gene-editing questions.

FIG. 14A illustrates a workflow of the exon suggestion feature within the guideRNA design module in CRISPR-GPT.

FIG. 14B illustrates a demonstration of the thought and action processes for exon suggestion in response to a real-world user request.

FIG. 15A illustrates a schematic representation of the LLM User-Proxy Agent workflow, where the agent gathers information from the user's input, interaction history, tool results, and current instructions to generate suggestions, and the user monitors task progression and reviews suggestions before sending the final decision to the Task Executor Agent for execution.

FIG. 15B illustrates examples of CRISPR-GPT auto-suggestions in response to various user inputs, where the state requests guide the user by asking for specific information (e.g., target species, Cas system, or delivery method), while CRISPR-GPT provides suggested answers and reasoning based on the provided inputs and context.

FIG. 16A illustrates an example of a generating AI tool for designing Homology Directed Repair (HDR) optimized sgRNAs for editing Hematopoietic Stem Cells (HSCs).

FIG. 16B-16D are graphs comparing results to experimental data at various genomic loci (CCR5 (16B), HBB (16C), and Sting1 (16D)) within HSCs using the HDR optimized sgRNAs.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to a large language model agent for automated design of gene-editing experiments. The system may provide assistance to researchers in planning and executing complex gene-editing tasks. In some cases, the system may utilize a combination of natural language processing, domain-specific knowledge, and external tools to guide users through various aspects of gene-editing experimental design.

The system may operate in three distinct modes to accommodate different user needs and experimental scenarios. In some cases, a Meta Mode may provide predefined workflows for common gene-editing tasks. An Auto Mode may offer more flexibility by generating customized task lists based on user inputs. A QA Mode may allow users to ask specific questions and receive targeted information throughout the experimental design process.

In some cases, the system may integrate domain knowledge from curated databases, published literature, and expert-designed protocols. This integration may enable the system to provide up-to-date and relevant guidance for gene-editing experiments. The system may also incorporate external tools and APIs to perform specialized tasks such as guide RNA design or off-target prediction.

The large language model agent may be designed to assist researchers across various stages of gene-editing experiments, including but not limited to selecting appropriate CRISPR systems, designing guide RNAs, choosing delivery methods, and planning validation experiments. By leveraging natural language interactions, the system may aim to make complex gene-editing techniques more accessible to researchers with varying levels of expertise.

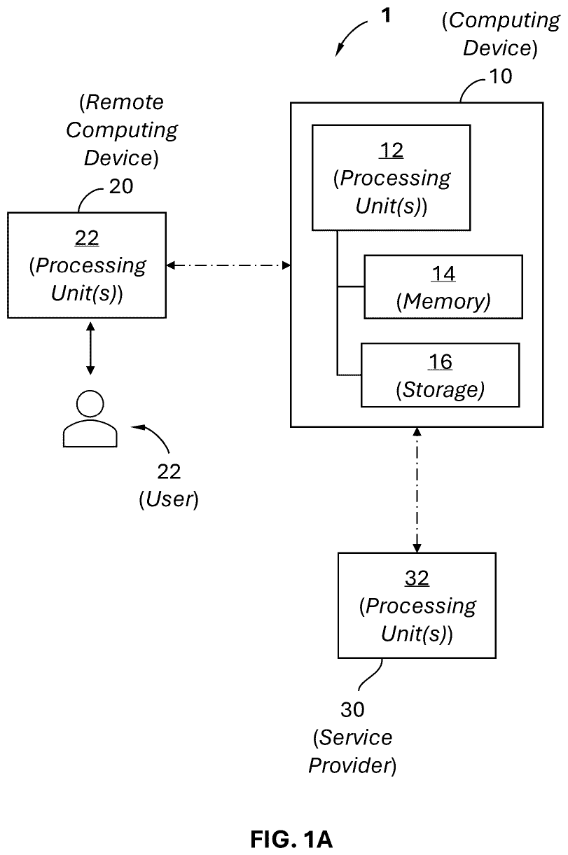

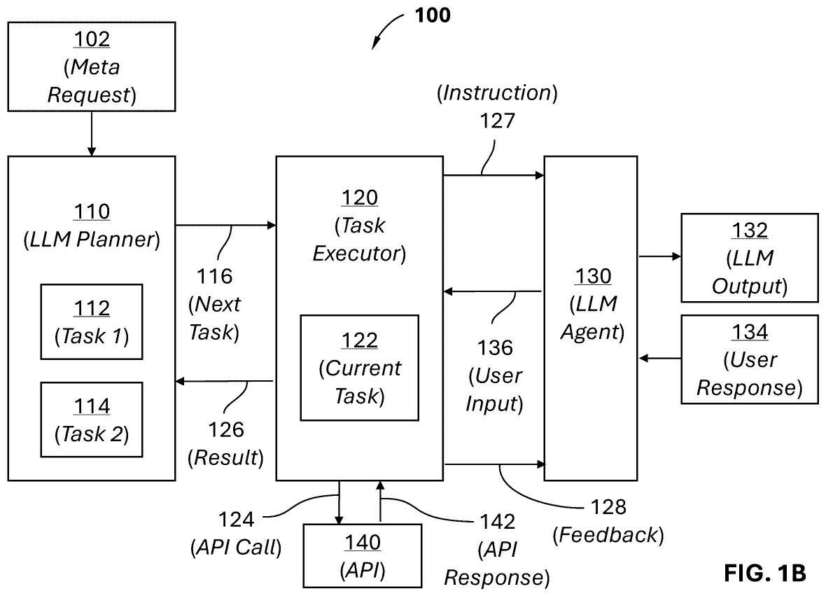

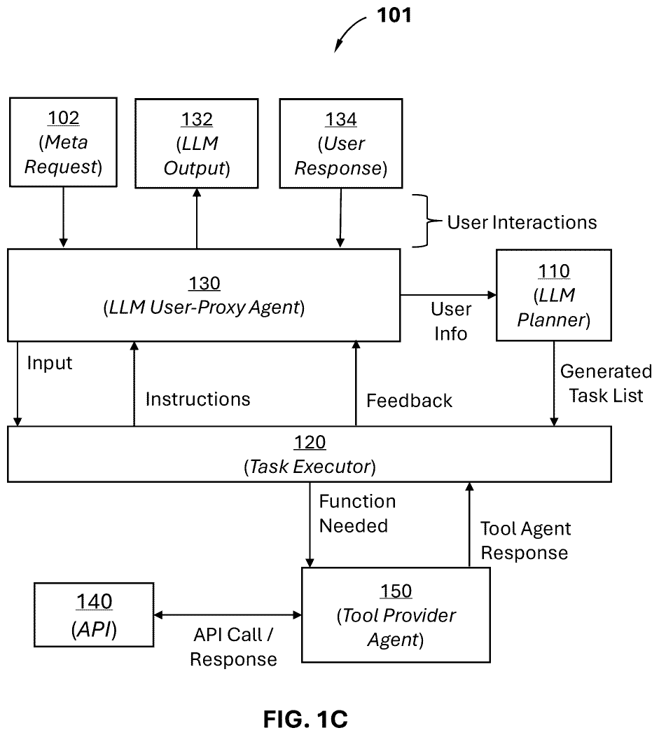

In some cases, a system for automated design of gene-editing experiments may include multiple components working together to process and execute gene editing requests. FIG. 1A illustrates a block diagram of such a system.

The system may include a computing device 1. In some cases, the computing device 1 may have a device housing 10. The device housing 10 may contain various components of the computing device 1. In some cases, the computing device 1 may include a processing unit 12. The processing unit 12 may be configured to execute instructions and perform computations necessary for the automated design of gene-editing experiments.

In some cases, the computing device 1 may also include a memory 14. The memory 14 may be operably coupled to the processing unit 12. The memory 14 may store data and instructions that may be accessed and executed by the processing unit 12. Additionally, the computing device 1 may include a storage 16. The storage 16 may provide non-transitory computer-readable storage for larger amounts of data and long-term storage of instructions.

The system may also include a remote computing device 20. In some cases, the remote computing device 20 may include a processing unit 22. The processing unit 22 may be configured to interact with a user, processing user inputs and providing outputs related to gene-editing experiment design.

In some cases, the system may further include a service provider 30. Such service providers may provide tools that can be utilized by the system. Such tools may be accessible via, e.g., API calls. The service provider 30 may utilize a processing unit 32. The processing unit 32 may be configured to provide additional computational resources or specialized services for gene-editing experiment design.

The computing device 1, remote computing device 20, and service provider 30 may be interconnected to enable communication and data flow between the devices. This interconnection may allow for distributed processing and storage capabilities in the automated design of gene-editing experiments.

In some cases, the system may include a reasoning framework. The reasoning framework may be configured to decompose a gene editing request into an ordered list of tasks. This decomposition may allow for systematic processing of complex gene-editing experiment designs.

The system may also include a Tool Provider module. In some cases, the Tool Provider module may be configured to connect to external APIs. This connection may allow the system to access additional tools and resources for gene-editing experiment design.

In some cases, the system may include a User-Proxy Agent module. The User-Proxy Agent module may be configured to interact with a user and process user inputs. This interaction may facilitate user-friendly operation of the system for automated design of gene-editing experiments.

The system may also include a Task Executor module. In some cases, the Task Executor module may be configured to implement tasks using state machines. This implementation may allow for structured execution of the tasks required for gene-editing experiment design.

In some cases, the non-transitory computer-readable storage device may contain instructions. When executed, these instructions may cause the system to perform methods for automated design of gene-editing experiments. The methods may include receiving a gene editing request, decomposing the request into tasks, sequentially executing the tasks, and outputting gene editing experiment design recommendations.

FIG. 1B illustrates a block diagram of a system 100 for automated design of gene-editing experiments. The system 100 may include various components that work together to process and execute gene editing requests.

In some cases, the system 100 may receive a meta request 102. The meta request 102 may include information about a requested gene-editing experiment. This information may be used to initiate the automated design process.

The system 100 may include an LLM planner 110. In some cases, the LLM planner 110 may be configured to decompose the meta request 102 into an ordered list of tasks. The LLM planner 110 may be connected to one or more task modules 112. These task modules 112 may represent different types of tasks that can be performed in the gene-editing experiment design process.

In some cases, the LLM planner 110 may be configured to sequentially send each task in the ordered list of tasks to a task executor 120. The LLM planner 110 may also optionally send a previous result to the task executor 120. The task executor 120 may be configured to implement tasks received from the LLM planner 110.

The task executor 120 may utilize state machines to decompose sub-goals. In some cases, the task executor 120 may consider a current task 122. The current task 122 may encapsulate a description of the task and any input required from a user.

The task executor 120 may be configured to connect to one or more external application programming interfaces (APIs) 140. In some cases, the task executor 120 may send an API call 124 to the API 140 and receive an API response 142. This interaction may allow the system 100 to access external tools and resources for gene-editing experiment design.

In some cases, the task executor 120 may be configured to provide instructions 127 to an LLM agent 130. The LLM agent 130 may act as a user-proxy agent, interacting with the user and processing user inputs. The task executor 120 may receive user input 136 from the LLM agent 130 responsive to the instructions 127.

The LLM agent 130 may generate LLM output 132, which may be presented to the user. In some cases, the LLM agent 130 may receive a user response 134. The user response 134 may be processed to generate the user input 136, which may be passed to the task executor 120.

The task executor 120 may be configured to provide feedback 128 to the LLM agent 130. This feedback 128 may be based on the task and/or user input 136. The feedback 128 may help guide the user through the gene-editing experiment design process.

In some cases, the LLM agent 130 may form a prompt based on the instruction 127 from the task executor 120, a request made by the user, a history of past interactions within the current task session, results from external APIs, or a combination thereof. The LLM agent 130 may use this prompt to determine an appropriate next action.

The system 100 may be configured to output one or more recommendations responsive to the meta request 102. These recommendations may be based on the processing performed by the various components of the system 100.

The components of the system 100 may work together to create a flow of information and processing. The LLM planner 110 may decompose the meta request 102 into tasks, which are then executed by the task executor 120. The task executor 120 may interact with external APIs 140 and the LLM agent 130 to gather necessary information and user input. The LLM agent 130 may facilitate user interaction and help determine appropriate actions based on the current state of the process. This flow of information and processing may enable the system 100 to provide automated assistance in designing gene-editing experiments.

As a simple example, a meta request 102 may be based on a user stating “I want to design sgRNA for knocking out human EGFR gene”. The LLM planner may identify two tasks in a workflow, such as “Cas System Selection (knockout)” and “sgRNA design (knockout)”. The first task may be sent to the Task Executor, which may send instructions 127 to the LLM agent containing “Cas System Selection guideline . . . ” The LLM agent may ask the user if they wish to minimize off-target effects. The user may indicate “Yes”. That user input is then sent back to the Task Executor. The Task Executor may check with one or more tools offered by a service provider, which may indicate the SpCas9 system may be optimal. The Task Executor may then generate the feedback of “we recommend the SpCas9 System” and pass that to the LLM agent for sending to the user. If the user agrees, that agreement may be sent to the Task Executor, and the Task Executor may send the result (e.g., “SpCas9”) to the LLM Planner, whereupon it may then send Task 2 to the Task Executor.

FIG. 1C illustrates a slightly different arrangement. There, the LLM User-Proxy Agent (e.g., LLM Agent 130) is configured to handle all user interactions. When appropriate, the LLM User-Proxy Agent sends user information to the LLM Planner (110). Based on that, the LLM Planner then sends a generated task list to the Task Executor (120). The LLM planner is charged with directing the entire workflow and breaking down the user's meta-request into a task chain. An example task chain may be, e.g., Task 1: CRISPR system selection; Task 2: Delivery methods selection; Task 3: guideRNA/pegRNA design; Task 4: Off-target prediction; Task 5: Experimental protocols; Task 6: Validation methods and assay primer design; and Task 7: Data analysis for editing outcomes. The Task Executor may take that generated task list and direct the subtasks needed to perform those tasks sequentially. The Task Executor may interact with the LLM User-Proxy Agent, and may interact with a Tool Provider Agent 150. The Tool Provider Agent may make interact with various APIs (either local APIs, or remote APIs) as needed to perform the required subtasks.

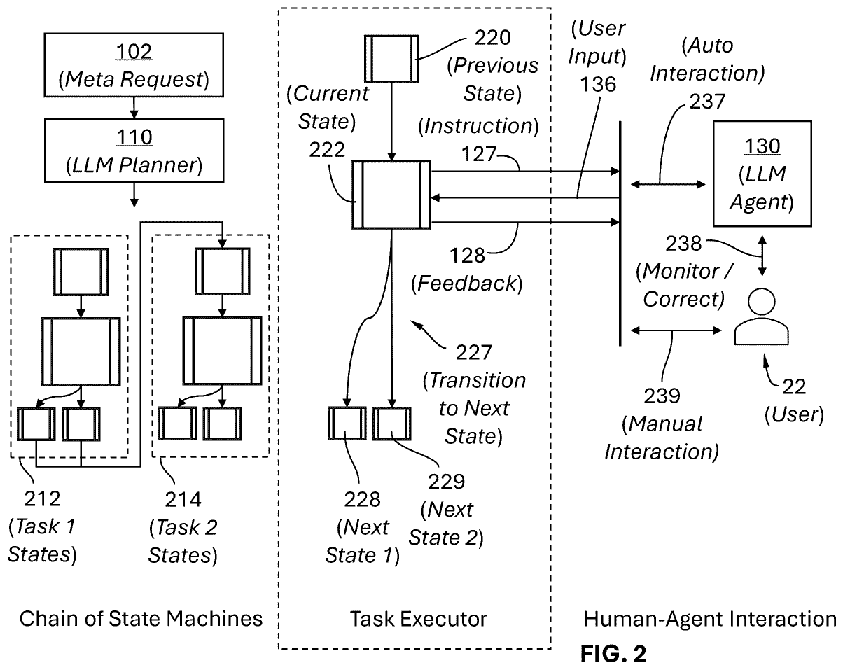

FIG. 2 illustrates a diagram showing a task decomposition process and state machine implementation for the system 100. The system 100 may utilize a structured approach to break down complex gene-editing requests into manageable tasks and states.

In some cases, the system 100 may receive a meta request 102 that provides input information about requested gene-editing experiments. The LLM planner 110 may process the meta request 102 and configure tasks based on the information provided. The LLM planner 110 may use a chain-of-thought reasoning technique to decompose the meta request 102 into an ordered list of tasks. This reasoning framework may comprise a large language model configured to analyze the meta request 102 and generate appropriate subtasks for execution.

The tasks generated by the LLM planner 110 may be associated with various states. For example, the system 100 may include task 1 states 212 and task 2 states 214. These states may represent different stages or aspects of each task that need to be addressed during the gene-editing experiment design process.

The system 100 may implement a state machine architecture to manage the progression through these tasks and states. In some cases, a current state 222 may receive input from a previous state 220. This flow of information between states may allow for continuity and context preservation throughout the task execution process.

There may be a transition 227 between states. The transition 227 may connect to a next state 1 228 and a next state 2 229. These next states may represent potential subsequent states based on the current processing and decision-making within the system 100.

The system 100 may provide multiple interaction pathways to accommodate different user preferences and experiment complexities. An auto interaction 237 may enable automated processing through the LLM agent 130. This pathway may allow for efficient handling of routine or well-defined tasks without requiring constant user input.

In some cases, a manual interaction 239 may allow for direct user input through the user input 136. This pathway may be useful for more complex or nuanced decisions that require human expertise or judgment.

The system 100 may also include a monitor and/or correct 238 function. This function may allow users to oversee the interactions and make corrections or adjustments as needed. The monitor and/or correct 238 function may help ensure accuracy and user control throughout the gene-editing experiment design process.

The task executor 120 may play a central role in implementing the state machine architecture. The task executor 120 may utilize state machines to decompose sub-goals of each task. This approach may allow for systematic progression through the various stages of gene-editing experiment design.

In some cases, the instruction 127 may be provided to the LLM agent 130 based on the current state 222. The LLM agent 130 may process these instructions to determine what user interaction may be required (e.g., provide certain information and ask a specific question, etc.). After receiving input from the user, the user input 136 may be provided from the LLM agent 130 to the task executor 120.

The task executor 120 may then process the user input 136 and provide feedback 128 as needed to the LLM agent 130. This feedback 128 may be based on the user input 136, and may be responsive to the desired output for a given step of a task, helping to guide the user through the gene-editing experiment design process.

The system architecture described in FIG. 2 may enable both automated and manual control flows, with the state machine implementation providing structured task decomposition and progress tracking. The components may work together to process gene-editing experiment requests and generate experimental design recommendations through a combination of automated reasoning and user interaction.

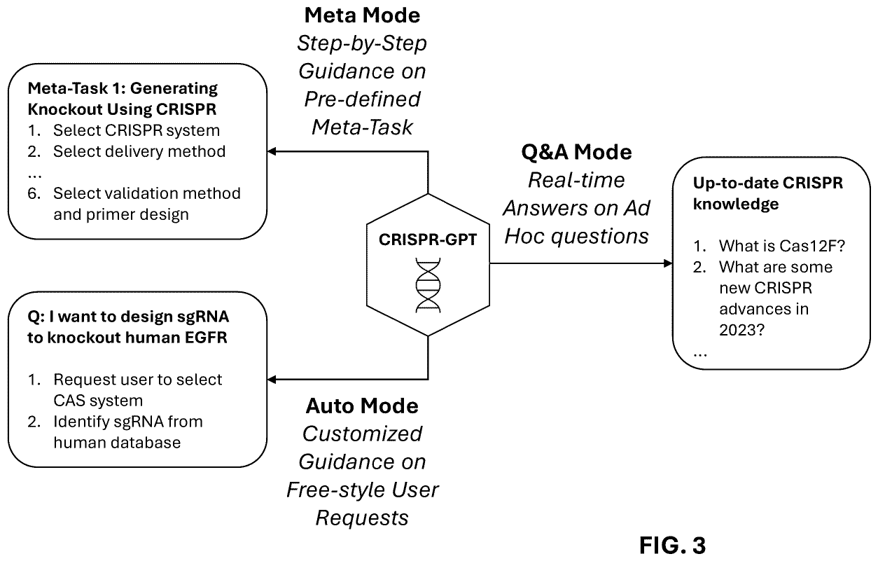

FIG. 3 illustrates a block diagram providing an overview of CRISPR-GPT's interactive modules for gene-editing experimental design. The system includes three main components: Q&A Mode, Meta Mode, and Auto Mode, each designed to assist users in different aspects of gene-editing experiment planning and execution.

In some cases, the system may receive a gene editing request from a user via a User-Proxy Agent module. The User-Proxy Agent module may facilitate interaction between the user and the system, allowing for natural language input and output.

The Q&A Mode component may provide real-time answers to questions about gene editing experiments and protocols. This mode may synthesize information from multiple sources, including a fine-tuned specialized model, retrieval-augmented generation, and a general large language model (LLM). The Q&A Mode may access an up-to-date CRISPR knowledge database containing information such as recent advances in CRISPR technology and domain-specific knowledge.

The Meta Mode component may provide step-by-step guidance on predefined meta-tasks through a state machine implementation. This mode may be useful for users who need assistance with common gene-editing workflows. The Meta Mode may connect to the CRISPR-GPT core through a step-by-step guidance pathway for predefined meta-tasks.

The Auto Mode component may provide customized guidance based on free-style user requests. This mode may offer more flexibility for users with specific or complex gene-editing experiment requirements. The Auto Mode may connect to the CRISPR-GPT core through a customized guidance pathway based on free-style user requests.

In some cases, the system may use a fine-tuned 8-billion-parameter LLM based on the Llama3-instruct model for enhanced problem-solving in gene-editing questions. This fine-tuned model may improve the system's ability to understand and respond to complex gene-editing queries.

The system may include a reasoning framework that decomposes the gene editing request into tasks. This decomposition may allow for a structured approach to experiment design, breaking down complex requests into manageable subtasks.

A Task Executor module may sequentially execute the tasks generated by the reasoning framework. The Task Executor may manage the progression through various stages of the experiment design process, ensuring that each task is completed in the appropriate order.

The system may process user inputs through these different modes and pathways to provide automated assistance with designing gene-editing experiments. The CRISPR-GPT component may coordinate the flow of information between the knowledge database and the different operational modes to generate appropriate responses and guidance for users.

In some cases, the system may output gene editing experiment design recommendations to the user via the User-Proxy Agent module. These recommendations may be based on the processing performed by the various components of the system, taking into account user inputs, task execution results, and information from the knowledge database.

The interactive modules of CRISPR-GPT, as illustrated in FIG. 3, may work together to provide a comprehensive solution for gene-editing experimental design. By offering multiple modes of interaction and leveraging advanced language models and knowledge databases, the system may assist researchers in planning and executing complex gene-editing tasks.

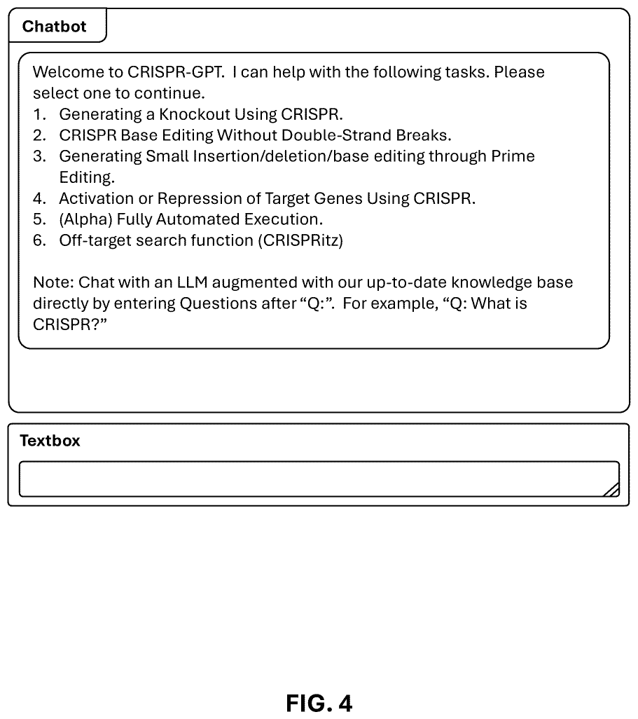

The system may include a user interface for interacting with the CRISPR-GPT agent. FIG. 4 illustrates an example user interface that may be used in some implementations of the system.

In some cases, the user interface may include a chatbot interface. The chatbot interface may display a welcome message to the user upon initiation of a session. This welcome message may introduce the capabilities of the CRISPR-GPT system and provide initial guidance to the user.

The chatbot interface may present a menu of available tasks related to gene editing experiments. In some cases, these tasks may include:
1. Generating a Knockout Using CRISPR.
2. CRISPR Base Editing Without Double-Strand Breaks.
3. Generating Small Insertion/deletion/base editing through Prime Editing.
4. Activation or Repression of Target Genes Using CRISPR.
5. (Alpha) Fully Automated Execution.
6. Off-target search function (CRISPRitz).

This menu may allow users to select specific gene editing tasks they wish to perform, providing a structured entry point for various experimental designs.

In some cases, the user interface may include a note indicating that users can engage in a question-and-answer mode. This mode may be activated by entering questions preceded by “Q:”. This feature may allow users to ask specific questions about CRISPR technology or experimental procedures, leveraging the system's knowledge base to provide relevant information.

The user interface may include a textbox component positioned below the main chat interface. This textbox may serve as the primary input mechanism for users, allowing them to enter text-based queries, select menu options, or provide responses to system prompts.

The chatbot interface may be designed to facilitate natural language interactions between the user and the CRISPR-GPT system. In some cases, the interface may display both user inputs and system responses in a conversational format, allowing for a dynamic and interactive exchange of information throughout the gene editing experimental design process.

In some cases, the system may implement a workflow for gene-editing experimental design. This workflow may include several steps to guide users through the process of selecting appropriate components and methods for their gene-editing experiments.

The workflow may begin with selecting a CRISPR system. In some cases, the method may extract parameters from user inputs related to gene editing. These parameters may be used to inform the selection of an appropriate CRISPR system for the user's specific needs.

Following CRISPR system selection, the workflow may proceed to selecting a delivery method. The method may perform a literature search based on the extracted parameters. In some cases, the method may rank candidate delivery methods using citations from the literature search results. The method may output a ranked list of candidate delivery methods.

In some cases, the method may include categorizing the user inputs into one of a plurality of predefined biological categories. The plurality of predefined biological categories may comprise specific categories related to gene editing applications, such as mammalian in vivo, mammalian embryos, mammalian primary cells or stem cells ex vivo, mammalian cell lines with strong evidence of high-efficiency transfection, mammalian cell lines or organoids without strong evidence of high-efficiency transfection, human in vivo or human embryos, and bacteria, viruses, and other organisms. The literature search may be performed based on the categorized biological category.

Ranking the candidate delivery methods may comprise retrieving a predefined set of delivery methods associated with the categorized biological category. The method may calculate a score for each delivery method based on the number of citations from the literature search results. The delivery methods may then be ordered based on the calculated scores.

The workflow may then proceed to designing sgRNA or pegRNA. In some cases, the method may receive a user request for guide RNA design. The method may extract relevant parameters from the user request. The method may access a pre-designed guide RNA table and apply a chain-of-table methodology to process the pre-designed guide RNA table based on the extracted parameters.

Applying the chain-of-table methodology may include selecting rows from the pre-designed guide RNA table where specified columns match given values. The method may order the selected rows based on values in a specified column. In some cases, the method may return a top number of rows from the ordered selection. The method may then select guide RNA sequences from the processed table and output the selected guide RNA sequences.

In some cases, the workflow may include evaluating off-target effects of the selected guide RNA sequences. The system may integrate external tools like CRISPRitz for this purpose. These tools may help assess the potential for unintended edits at non-target genomic locations.

The workflow may also include selecting experimental protocols. In some cases, the system may use a curated knowledge base of peer-reviewed literature for retrieval-augmented generation. This may allow the system to provide up-to-date and relevant protocol suggestions based on the specific parameters of the user's experiment.

For validation methods, the workflow may include steps for designing primers and selecting appropriate sequencing or detection methods. The system may integrate external tools like Primer3 for primer design tasks. In some cases, the system may include an exon suggestion module for guideRNA design that considers important functional domains of genes. This may help ensure that the designed experiments target relevant regions of the gene of interest.

Throughout the workflow, the system may provide guidance and recommendations based on the specific inputs and requirements of the user's gene-editing experiment. The integration of literature-based ranking, chain-of-table methodologies, and external tools may help users make informed decisions at each step of the experimental design process.

In some cases, the system may evaluate the performance of CRISPR-GPT compared to other language models. The evaluation may use various metrics to assess the effectiveness of CRISPR-GPT across different operational modes.

The system may obtain a dataset of gene editing discussions from a public forum. This dataset may be preprocessed to extract question-answer pairs. The preprocessing may include anonymizing personal information in the discussions to protect user privacy. In some cases, the preprocessing may involve extracting question-answer pairs from individual discussion threads. The system may filter the extracted pairs to remove irrelevant or low-quality content, ensuring a high-quality dataset for training and evaluation.

The system may fine-tune a pre-trained language model using the extracted question-answer pairs. In some cases, the fine-tuning process may use full parameter fine-tuning, where all parameters of the pre-trained model are updated during training. Alternatively, the system may employ quantized low-rank adaptation (QLoRA) fine-tuning, which may reduce the computational requirements while maintaining performance. The choice between full parameter fine-tuning and QLoRA may depend on factors such as available computational resources and desired model performance.

After fine-tuning, the system may store the fine-tuned model for subsequent use in gene editing tasks. This may allow for efficient retrieval and application of the model in various gene editing scenarios.

The system may evaluate the fine-tuned model using a test set of gene editing questions. This evaluation may involve comparing the performance of the fine-tuned model to the pre-trained model on the test set. The comparison may help assess the effectiveness of the fine-tuning process and determine whether the fine-tuned model offers improved performance in gene editing-related tasks.

In some cases, the evaluation may consider multiple performance metrics. These metrics may include accuracy, which may measure the correctness of the model's responses. The system may also assess reasoning ability, which may evaluate the model's capacity to provide logical explanations and justifications for its answers. Completeness may be another metric, potentially measuring how thoroughly the model addresses all aspects of a given question. Additionally, the system may evaluate conciseness, which may assess the model's ability to provide succinct and relevant responses without unnecessary information.

The system may perform evaluations across different operational modes, such as Meta Mode, Auto Mode, and QA Mode. This multi-mode evaluation may help assess the model's performance in various gene editing scenarios and user interaction patterns.

In some cases, the evaluation results may indicate that CRISPR-GPT outperforms other language models in specific gene editing tasks. The performance improvements may be particularly noticeable in areas requiring specialized knowledge of gene editing techniques and experimental design.

The system may use the evaluation results to further refine and improve the CRISPR-GPT model. This iterative process of evaluation and improvement may help ensure that the model remains up-to-date and effective in assisting with gene editing experimental design tasks.

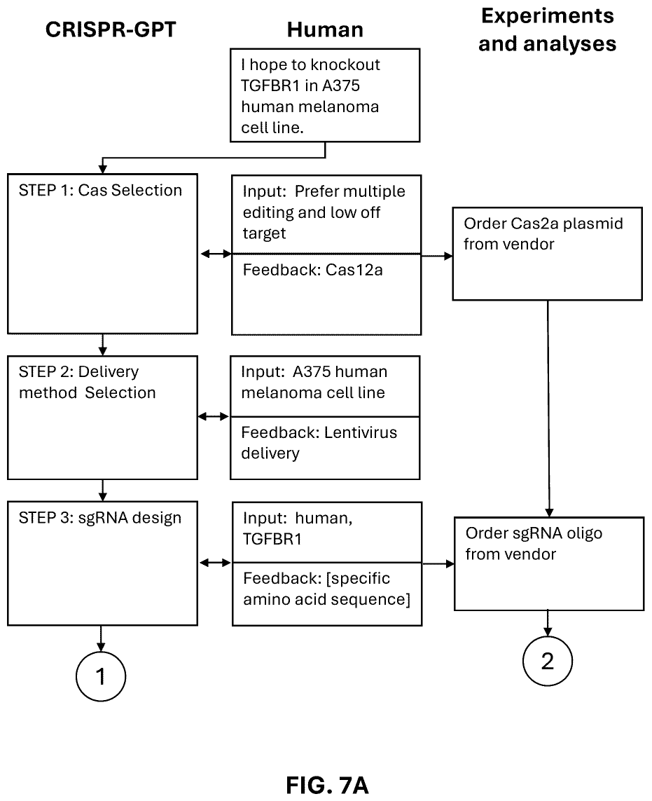

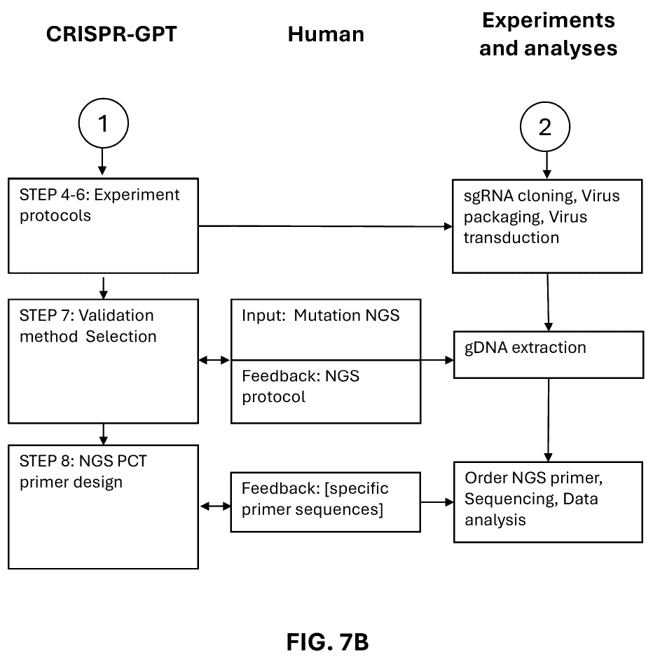

In some cases, the system may demonstrate real-world application through gene knockout experiments. FIG. 7A and FIG. 7B illustrate an example workflow for designing and implementing gene-editing experiments using CRISPR-GPT.

The method may receive a gene editing query from a user. For example, as shown in FIG. 7A, a user may input “I hope to knockout TGFBR1 in A375 human melanoma cell line.” The system may process this query using a model trained with fine-tuning on gene editing discussions.

To address the query, the method may retrieve relevant information from a curated knowledge base of gene editing literature. This retrieval process may include embedding the gene editing query and documents in the knowledge base into semantic vectors. The method may then perform a similarity search to identify the most relevant documents based on cosine similarity between the query vector and document vectors.

After identifying relevant documents, the method may summarize the identified relevant documents in relation to the gene editing query. This summarization may help distill key information pertinent to the user's specific experimental goals.

The method may synthesize an answer based on the processed query and retrieved information. This synthesis may include combining information from the processed query, the retrieved relevant information, and a response generated by a fine-tuned large language model trained on gene editing discussions. The synthesis process may aim to generate a concise answer that addresses the specific aspects of the gene editing query.

As illustrated in FIG. 7A, the system may guide the user through several key steps in designing the gene knockout experiment. These steps may include Cas selection, delivery method selection, and sgRNA design. For each step, the system may provide recommendations based on the synthesized information and user preferences.

For example, when the user expresses a preference for multiple editing and low off-target effects, the system may recommend using Cas12a. Similarly, based on the specified cell line (A375 human melanoma), the system may suggest lentivirus delivery as an appropriate method.

FIG. 7B shows the continuation of the experimental workflow, including steps for implementing the designed experiment and analyzing the results. The method may output synthesized answers at various stages of this process, providing guidance on experimental protocols, validation methods, and data analysis techniques.

In some cases, the system may recommend next-generation sequencing (NGS) for mutation detection and validation of the knockout. The method may provide detailed protocols for gDNA extraction, PCR primer design, and sequencing library preparation.

Throughout this process, the system may continue to retrieve and synthesize relevant information from its knowledge base, ensuring that the recommendations and protocols provided are up-to-date and tailored to the specific experimental parameters.

By guiding users through this comprehensive workflow, from initial query to final data analysis, the system may demonstrate its capability to assist in real-world gene editing applications, leveraging its trained model and curated knowledge base to provide relevant, concise, and specific guidance at each step of the experimental process.

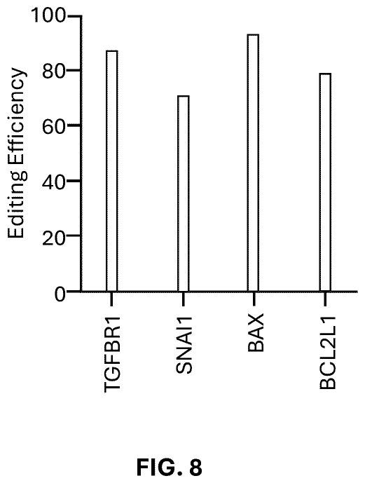

In some cases, the system may evaluate the editing efficiency of gene knockout experiments designed using CRISPR-GPT. FIG. 8 illustrates a bar graph showing editing efficiency values for four different gene targets: TGFBR1, SNAI1, BAX, and BCL2L1.

The editing efficiency results depicted in FIG. 8 demonstrate varying levels of success across the four target genes. For TGFBR1, the graph indicates an editing efficiency of approximately 85%. SNAIL shows a lower efficiency of around 70%. BAX exhibits the highest editing efficiency among the four targets, reaching approximately 90%. BCL2L1 displays an editing efficiency of about 80%.

These results may provide insights into the effectiveness of the CRISPR-GPT system in designing gene knockout experiments. The high editing efficiencies observed, particularly for BAX and TGFBR1, may suggest that the guide RNA sequences and experimental protocols recommended by CRISPR-GPT are capable of facilitating efficient gene editing.

The variation in editing efficiencies across different gene targets may reflect inherent differences in the accessibility or susceptibility of these genomic regions to CRISPR-mediated editing. Factors such as chromatin structure, DNA sequence context, or gene expression levels may influence the editing efficiency for each target.

In some cases, the editing efficiency data may be used to further refine and improve the CRISPR-GPT system. By analyzing the characteristics of targets with higher editing efficiencies, the system may potentially identify patterns or features that contribute to successful gene editing. This information may be incorporated into future iterations of the system to enhance its ability to design effective gene knockout experiments across a wide range of targets.

The editing efficiency results presented in FIG. 8 may serve as a validation of the CRISPR-GPT system's capability to assist in real-world gene editing applications. By achieving high editing efficiencies for multiple gene targets, the system may demonstrate its potential utility in facilitating successful gene knockout experiments in various research contexts.

As will be understood, disclosed are methods for training models to perform the disclosed functions. In addition, such models may then be used for inference to actually perform those functions as disclosed herein.

### Examples

Genome engineering technology has revolutionized biomedical research by enabling precise genetic modifications. However, designing effective gene-editing experiments requires a deep understanding of both the CRISPR technology and the biological system involved. Meanwhile, despite their versatility and promise, Large Language Models (LLMs) often lack domain-specific knowledge and struggle to accurately solve biological design problems. In this work, we present CRISPR-GPT, an LLM agent system to automate and enhance the CRISPR-based gene-editing design process. CRISPR-GPT leverages the reasoning capabilities of LLMs for complex task decomposition, decision-making, and interactive human-AI collaboration. This system is driven by multi-agent collaboration, and it incorporates domain expertise, retrieval techniques, external tools, and a specialized LLM fine-tuned with a decade's worth of open-forum discussions among gene-editing scientists. CRISPR-GPT assists users in selecting CRISPR systems, experiment planning, designing gRNAs, choosing delivery methods, drafting protocols, designing assays, and analyzing data. We showcase the potential of CRISPR-GPT in assisting beginner researchers with gene-editing from scratch, knocking-out four genes with CRISPR-Cas12a in a human lung adenocarcinoma cell line and epigenetically activating two genes using CRISPR-dCas9 in human melanoma cell line, both successful on first attempt. CRISPR-GPT enabled fully AI-guided gene-editing experiment design across different modalities, validating its effectiveness as an AI co-pilot in genome engineering.

Large language models (LLMs) have demonstrated exceptional capabilities in language skills and encapsulate a tremendous amount of world knowledge. Recent research has also enhanced LLMs with external tools, improving their problem-solving abilities and efficiencies. Moreover, LLMs have also demonstrated potential as tool makers and black-box optimizers. To this end, researchers have explored LLM-based specialized models for various scientific domains, particularly for mathematics and chemistry tasks. ChemCrow, for example, uses tool-augmented LLM for solving a range of chemistry-related tasks such as paracetamol synthesis, whereas Coscientist integrated automated experimentation, achieving successful optimization of palladium-catalyzed cross-coupling reaction. LLMs have also shown initial promise in generating biological protocols, as demonstrated by studies like BioPlanner. While recent advancements, such as OpenAI's o1 preview, have improved reasoning abilities in areas like mathematics and coding, progress in biological tasks remains comparatively limited. This limitation stems from general-purpose LLMs' lack of in-depth understanding of biology, compounded by the unique challenges of biological experiments, including the variability of living systems, the noisy nature of biological data, and the highly specialized, less transferable nature of biological skills and tools.

Gene editing has transformed biological research and medicine, allowing for precise DNA modifications for both therapeutic and experimental applications. CRISPR-Cas, the most well-known gene-editing technology, originated from bacterial immune systems. Its development has led to advanced techniques like CRISPR activation and interference (CRISPRa/i), base-editing, and prime-editing, creating a powerful toolkit for genetic modification and epigenetic modulation. In basic biomedical research, CRISPR gene-editing has become one of the most frequently used laboratory techniques: at the largest non-profit plasmid DNA repository, Addgene, 8 of the 15 top requested plasmids worldwide were for CRISPR gene-editing. On the application side, CRISPR has produced the first permanent cure for Sickle Cell Disease (SCD) and β-thalassemia, as well as facilitating plant engineering for sustainable agriculture. As one of the most powerful biotechnologies, numerous software and protocols exist for specific gene-editing tasks. Despite these resources, designing an end-to-end solution—from CRISPR-Cas system selection, gRNA design, off-target evaluation, to delivery and data analysis—remains complex, particularly for newcomers. AI-assisted tools can simplify gene-editing experiment design, making the technology more accessible and accelerating scientific and therapeutic discoveries.

Biological research presents unique challenges due to its complexity and variability. While tool-augmented LLMs have proven effective in certain tasks, advanced areas of biology such as gene-editing require specialized LLMs. Such models must integrate accurate domain knowledge and generate experimentally viable solutions, possessing the intelligence and automation to enable complex decision-making, navigate less well-defined situations, and perform problem-solving and troubleshooting.

Disclosed is CRISPR-GPT, a solution that combines the strengths of LLMs with domain-specific knowledge, chain of thought reasoning, instruction finetuning, retrieval techniques and tools. CRISPR-GPT is centered around LLM-powered planning and execution agents (see FIG. 1B). This system leverages the reasoning abilities of general-purpose LLMs and multi-agent collaboration for task decomposition, constructing state machines, and automated decision-making (see FIG. 2). It draws upon expert knowledge from leading practitioners and peer-reviewed published literatures in gene-editing for retrieval-augmented generation (RAG).

To make CRISPR-GPT “think” more like a scientist, the system is augmented with CRISPR-Llama3, a new specialized 8B-parameter LLM which the inventors have fine-tuned on ten years' worth of scientific discussions among gene-editing experts around the world. This fine-tuned LLM enhances the agent's problem-solving skills and provides brainstorming second opinions on difficult inquiries.

CRISPR-GPT has integrated a variety of search and bioinformatics tools, including, but not limited to, Google web search, Primer3, CRISPRitz for off-target prediction, CRISPresso2 for next-generation sequencing (NGS) data analysis. It also leverages public gRNA libraries, published papers and protocols to provide users with optimized gene-editing strategies.

The example CRISPR-GPT supports four major gene-editing modalities and 22 gene-editing experiment tasks. It offers tunable levels of automation via three modes: Meta, Auto, and QA. They are designed to accommodate users from novice PhD-level scientist fresh to gene-editing, to domain experts looking for more efficient, automated solutions for selected tasks. “Meta Mode” is designed for beginner researchers, guiding them through a sequence of essential tasks from selection of CRISPR systems, delivery methods, to designing gRNA, assessing off-target efficiency, generating experiment protocols and data analysis. Throughout this decision-making process, CRISPR-GPT interacts with users at every step, provides instructions, and seeks clarifications when needed. “Auto Mode” caters to advanced researchers and does not adhere to a predefined task order. Users submit a free-style request, and the LLM-planner decomposes this into tasks, manages their interdependence, builds a customized workflow and executes them automatically. It fills in missing information based on initial inputs and explains its decisions and thought process, allowing users to monitor and adjust the process. “Q&A Mode” supports users with on-demand scientific inquiries about gene-editing.

To assess the AI agent's capabilities to perform gene-editing research, we compiled an evaluation test set, Gene-editing-Bench, from both public sources and human experts. This test set covers a variety of gene-editing tasks. By using the test set, extensive evaluation of CRISPR-GPT's capabilities were performed in major gene-editing research tasks, such as experiment planning, delivery selection, sgRNA design, and experiment troubleshooting. Additionally, human experts were invited to perform a thorough user experience evaluation of CRISPR-GPT and collected valuable human feedback.

Further, CRISPR-GPT can be implemented in real-world wet labs. Using CRISPR-GPT as an AI co-pilot, a fully AI-guided knockout of four genes-TGFBR1, SNAI1, BAX, and BCL2L1-using CRISPR-Cas12a in human lung adenocarcinoma cell line, as well as AI-guided CRISPR-dCas9 epigenetic activation of two genes—NCR3LG1, CEACAM1—in a human melanoma model was performed. All wet-lab experiments were carried by junior researchers not familiar with gene-editing. They both succeeded on the first attempt, confirmed by not only editing efficiencies, but also biologically relevant phenotypes and protein-level validation, highlighting the potential of LLM-guided biological research.

Mindful of the ethical and safety considerations for gene-editing-especially in human applications, several safeguards were implemented to prevent dual usages and protect user privacy. These include restrictions on human heritable gene-editing or pathogen engineering, measures to ensure the privacy of user-provided genetic information, and alerts for potential unintended consequences, reflecting our commitment to responsible use in alignment with the broader scientific and ethical discourse on gene-editing technologies.

The CRISPR-GPT consists of the following 4 core components (FIG. 1): LLM planner, Tool providers, Task executors, and the LLM User-Proxy Agent that serve as the interface with users for taking inputs and communicating outputs. Each component can be viewed as an LLM-powered single agent with relatively simple functionality, and the overall system functions via multi-agent interaction. These single agents leverage general-purpose LLMs, such as GPT-40 (used in all four core agents unless otherwise specified), as their base model to handle a wide range of tasks. The LLMs rely on carefully designed prompts to guide their behavior and interactions.

Example prompts are discussed below.

The following prompt format was used for task decomposition for the LLM planner in the automation mode. The LLM planner interprets the user's request and decomposes it into a list of tasks. The LLM planner is prompted to respect the task dependencies stated in the Task Description Table.

### Prompt:

```
Please act as an expert in CRISPR technology. Given the user input, think step by step and
  generate a list of tasks for execution. First refer to the task description table below, and try
  to figure out if the user needs to directly jump into a task, or the user needs to complete
  several tasks. Make sure to respect the task dependencies and include all dependent tasks in
  the list.
Please format your response and make sure it is parsable by JSON.
## Task Description Table
{Task Description Table}
## Demonstrations:
If the user only needs to design guideRNA for knockout, then return [’knockout.StateStep1’,
  ’knockout.StateStep3’]. Reason: this directly matches knockout.StateStep3. But it needs to
  complete knockout.StateStep1 first, so both ’knockout. StateStep1’ and
  ’knockout.StateStep3’ are returned.
User Input:
″{user_message}″
Response format:
{{
″Thoughts″: ″<thoughts>″,
″Tasks″: [″<task1>″, ″<task2>″] ## a list of task names
}}
```

The task description table contains all the implemented tasks and their dependencies; see Table 1 for details.

### Additional Prompt Detail:

```
For knockout
task name: task descriptions: dependency
knockout.StateStep1: Cas System selection for knockout : none
knockout.StateStep2: Delivery approach selection for knockout : none
knockout.StateStep3: guideRNA design for knockout : needs to complete
  knockout.StateStep1 first
knockout.StateStep4: Experimental Protocol Selection for knockout : needs to
  complete knockout.StateStep2 first
knockout.StateStep4_5_1_Sanger: Primer Design for knockout, Mutation
  sequencing by Sanger : none
knockout.StateStep4_5_1_NGS: Primer Design for knockout, Mutation
  sequencing by next-generation sequencing (NGS): none
For base editing
task name: task descriptions: dependency
base_editing.StateStep1: Base Editor System selection for base editing : none
base_editing.StateStep2: guideRNA design for base editing : needs to complete
  base_editing.StateStep1 first
base_editing.StateStep3: Delivery approach selection for base editing : none
base_editing.StateStep4: Experimental Protocol Selection for base editing :
  needs to complete base_editing.StateStep3 first
base_editing.StateStep4_5_1_Sanger: Primer Design for base editing,
  Mutation sequencing by Sanger : none
base_editing.StateStep4_5_1_NGS: Primer Design for base editing,
  Mutation sequeuncing by next-generation sequencing (NGS) : none
For prime editing
task name: task descriptions: dependency
prime_editing.StateStep1: Prime Editing System selection for prime editing : none
prime_editing.StateStep2: Delivery approach selection for prime editing : none
prime_editing.StateStep3: pegRNA design for prime editing: needs to complete
  prime_editing.StateStep1 first
prime_editing.StateStep4: Experimental Protocol Selection for prime editing :
  needs to complete prime_editing.StateStep2 first
prime_editing.StateStep4_5_1_Sanger: Primer Design for prime editing,
  Mutation sequencing by Sanger : none
prime_editing.StateStep4_5_1_NGS: Primer Design for prime editing,
  Mutation sequencing by next-generation sequencing (NGS) : none
For CRISPRa/CRISPRi
task name: task descriptions: dependency
act_rep.StateStep1: Activation or repression system selection
for CRISPRa/CRISPRi : none
act_rep.StateStep2: Delivery approach selection for CRISPRa/CRISPRi : none
act_rep.StateStep3: guideRNA design for CRISPRa/CRISPRi : needs to complete
  act_rep.StateStep1 first
act_rep.StateStep4: Experimental Protocol Selection for CRISPRa/CRISPRi : needs
  to complete act_rep.StateStep2 first
act_rep.StateStep4_5_1: Primer Design for CRISPRa/CRISPRi, qPCR : none
For Off-Target Prediction
task name: task descriptions: dependency
off_target.StateStep1: Off-target search/prediction using CRISPRitz: none
```

The relevant information is synthesized into {system_message}, including the instruction of the current state, the interaction history between the agent and the system, and potentially the results from external tools and libraries. Next, the meta request of the user is supplied in {meta_prompt}. Then the LLM-agent is prompted to understand the current state and make decisions on behalf of the user.

```
Please act as you are using the CRISPR design tool. Given the user meta request, the current
  inquiry provided by the tool, think step by step and generate an answer to the questions.
  Please format your response and make sure it is parsable by JSON.
Rules:
1. Answer the inquiry directly on behalf of the user. Don't raise any additional question to the
  user.
2. If the inquiry is a multiple-choice question, then directly output one choice.
3. If the inquiry asks you to supply any gene sequence, then answer the question with “I don't
  know” and let the user take manual control.
User Meta Request:
“{meta_prompt}”
Current Inquiry:
“{system_message}”
Response format:
{{
“Thoughts”: “<thoughts>”,
“Answer”: “<response string>”
}}
```

TABLE 1
List of meta-mode tasks (4 major meta-tasks and 22 specific tasks)

| Meta-Tasks | Gene editing scenarios | Individual design tasks |
|---|---|---|
| CRISPR | Single/multiple genes | CRISPR/Cas system selection |
| Knockout | knockout, deletion of | Delivery method selection |
| | gene fragments | sgRNA design for knockout |
| | | off-target evaluation |
| | | experimental protocol recommendation |
| | | validation protocol recommendation |
| | | and primer design for sequencing |
| CRISPR | Gene activation and | CRISPR/Cas Activation/Interference |
| activation/ | repression | system selection |
| interference | | Delivery method selection |
| | | sgRNA design for activation/interference |
| | | off-target evaluation |
| | | experimental protocol recommendation |
| | | validation protocol recommendation |
| | | and primer design for qPCR |
| CRISPR | Single base replacement | Base editing system selection |
| Base | from CG to AT or AT | Delivery method selection |
| Editing | to CG and broad | sgRNA design for base editing |
| | mutagenesis | off-target evaluation |
| | | experimental protocol recommendation |
| | | validation protocol recommendation |
| | | and primer design for sequencing |
| CRISPR | Small fragment, | Prime editing system selection |
| Prime | insertion, replacement, | Delivery method selection |
| Editing | and deletion | pegRNA design for prime editing |
| | | off-target evaluation |
| | | experimental protocol recommendation |
| | | validation protocol recommendation |
| | | and primer design for sequencing |

Task Executor operates as state machines, providing robust decomposition and progress control. A total of 22 tasks were implemented, each decomposed into sub-goals, with states providing instructions and guiding users through decision-making via multiple rounds of textual interaction. A central management class tracks the current state, task queue, memory for state outputs, and execution history. State transitions occur sequentially, or based on conditional logic from execution results as needed. States process user input and generate structured outputs containing the status, reasoning, and response, which are stored persistently to ensure continuity across tasks. This framework supports both predefined workflows (Meta Mode) and dynamically generated task sequences (Auto Mode), offering flexibility, reliability, and robust error handling for executing CRISPR-GPT tasks.

In Meta Mode, the Task Executor follows predefined workflows that cover complete pipelines for four Meta Tasks, each corresponding to a major type of gene-editing experiment. In Auto Mode, the LLM planner dynamically generates a customized sequence of tasks based on the user's meta-request. The Task Executor then constructs and executes the workflow by chaining the state machines of the corresponding tasks into a larger state machine, enabling seamless and automated execution of complex gene-editing pipelines.

To connect language models with external functionalities, the system needs to (1) analyze the current situation and judge whether it's suitable to call an external tool; (2) know what kinds of tools are available and choose the best from them. Instead of directly exposing the interfaces of the APIs to LLMs, in CRISPR-GPT, the usage of APIs is wrapped inside the states and expose more user-friendly and LLM-friendly textual interfaces through hand-written instructions and responses. In plain words, users (human agents & LLM user-proxy agents) are taught to use the tools. The tools include, e.g., Google web search, Google Scholar search, literature retrieval, and bioinformatic tools like Primer3, CRISPRitz, CRISPResso2.

### LLM Planner Automatically Plans Gene-Editing Experiments Based on the User's Request

Large Language Models (LLMs), such as GPT-4, Gemini, and Claude, serve as the reasoning core of the LLM-powered agent to solve real-world decision-making problems. Our LLM planner operates based on two key components: (1) the user query and (2) a predefined table containing comprehensive descriptions and interdependency information for all available tasks. Using the ReAct prompting technique, the LLM is prompted to output a chain-of-thought reasoning path along with the final action from the plausible action set. Based on the LLM's internal knowledge, combined with our manually written task descriptions and decomposition instructions, the planner analyzes the user's request, intelligently decomposes it into an ordered list of tasks, and ensures the dependencies between tasks are respected. Once the decomposition is complete, the corresponding state machines are automatically chained together to execute all tasks in the appropriate sequence. For robustness in this example, the LLM was prevented from dynamically adding or deleting tasks (state machines) during execution. However, it is acknowledged that enabling dynamic task management is an important step toward developing a more intelligent science AI agent.

The LLM User-Proxy Agent automatically interacts with the Task Executor based on the meta request. Central to our system is the LLM User-Proxy Agent, which acts as an intermediary between the user and a state machine. This state machine is derived from an initial task decomposition step, effectively breaking down the gene-editing process into a structured sequence of actions and decisions. At each step in this sequence, the state machine presents a current state to the LLM agent, which encapsulates a description of the task at hand and specifies any required input to move forward. The required input varies depending on the task type. For example, tasks may require information such as the general experimental context (e.g., I hope to design 4 sgRNAs targeting human TGFBR1) or the specific Cas system (e.g., enCas 12a).

The LLM User-Proxy Agent's role is to interpret the current state and make informed decisions on behalf of the user. To achieve this, the agent integrates multiple sources of information, including:
Current state instructions provided by the state machine,
Specific requests made by the user,
Interaction history within the current task session, and
Results from external computational tools integrated into the system.

This synthesized information is formatted into a structured prompt for the LLM User-Proxy Agent, which then determines the most appropriate next action. For example, when designing a CRISPR experiment, the agent might combine a user's input about a target gene with computational results identifying suitable sgRNA candidates to propose the next step in the workflow.

While the User-Proxy Agent operates autonomously, user oversight is integral to the system. Users are encouraged to monitor task progression and interact with the agent as needed to ensure accuracy. If any errors or misinterpretations occur, users can quickly intervene and provide corrections, maintaining the integrity of the gene-editing experiment design.

This approach emphasizes a collaborative synergy between human expertise and artificial intelligence. By leveraging the LLM agent's ability to process and act on complex information, we enable a more efficient, accurate, and user-friendly experience in designing CRISPR gene-editing experiments. The sequential decision-making framework not only streamlines task execution but also ensures that user input remains a cornerstone of experiment planning and design.

### Delivery Method Selection Agent

This disclosed approach mirrors the thought process of human gene-editing experts to identify the most appropriate delivery method based on the user's specific biological system. The workflow begins by instructing the LLM to extract key biological terms from the user's natural language request. These terms provide insight into the biological context of the experiment. The LLM is then tasked with accessing up-to-date information using a Google Search API to gather additional context about the biological system in the user request.

Based on the combined information from the user's request and external data, the LLM categorizes the system into one of seven major biological categories:
1. Mammalian in vivo.
2. Mammalian embryos.
3. Mammalian primary cells or stem cells ex vivo.
4. Mammalian cell lines with strong evidence of high-efficiency transfection.
5. Mammalian cell lines or organoids without strong evidence of high-efficiency transfection.
6. Human in vivo or human embryos.
7. Bacteria, viruses, and other organisms.

These categories encompass the majority of biological systems relevant to CRISPR delivery. For each category, 1-3 delivery methods were curated based on human experts' knowledge, which represent the most commonly used CRISPR delivery strategies.

To further tailor the recommendations to the user's specific scenario, the agent system conducts a Google Scholar search to identify relevant peer-reviewed literature. The search is guided by the key terms extracted from the user's request. From the search results, the top 10 relevant papers are ranked by citation count, providing a quantitative measure for prioritizing the potential delivery options within each biological category.

While citation numbers are not a definitive metric for determining the most appropriate delivery method, they offer a useful reference point. This approach helps to present well-informed recommendations along with relevant literature to the user.

gRNA Design Agent.

Designing sgRNAs is a critical challenge in CRISPR editing, as it directly influences editing efficiency. Numerous sgRNA design tools (both web-based and software packages) are currently available, each following general design principles and utilizing various metrics—such as on-target and off-target prediction scores, exon number, and cut position—to rank the designed sgRNAs. We identified two major challenges for users: (1) finding a trustworthy source for sgRNA design and (2) efficiently selecting sgRNAs that meet their specific requirements without having to assess every individual metric.

To address these challenges, predesigned sgRNA tables from CRISPick, a highly reputable and widely-used pre-designed sgRNA library from the Broad Institute, were utilized. This resource has been extensively validated and employed by scientists globally. The reasoning and action (ReAct) capabilities of large language models (LLMs) was harnessed to process table queries based on user inputs. The disclosed agent performs a series of actions to process the tables step-by-step to generate the results, akin to a recently published “chain-of-table” methodology.

The agent system can choose from four key functions:

SELECT: Retrieves rows where the specified column matches the given value.

BETWEEN: Selects rows where the specified column's values fall between a specified range (inclusive).

ORDERBY: Orders the table based on values in a specified column.

TOP: Returns the top N rows of the table.

These functions can be expanded in the future, either by human input or through LLM-generated suggestions. The agent simultaneously extracts relevant parameters from the user's request and the table, then uses these functions and parameters to collect and present the pre-designed sgRNAs along with relevant information. The results are provided to users through a table visualization and a download link.

Additionally, an optional Exon Suggestion module was developed within the sgRNA design function, currently applicable only for CRISPR Knockout sgRNA design. It has been reported that sgRNAs targeting non-essential regions of genes may be less effective. For instance, Shi et al. demonstrated that targeting only the BD1/BD2 domains effectively disrupted the BRD4 gene function. It was hypothesized that, given the vast knowledge base of general LLMs, they could suggest important functional domains (exons) for genes of interest. The LLM was prompted to reason through the functional domains of the user's target genes and provide recommendations on potentially relevant exons. This information was then integrated into the table queries.

Currently, there aren't any available sgRNA design tools that could take specific gene function domains into consideration, and it is believed this exon suggestion feature provides a valuable reference for the users. In the meantime, it is acknowledged that the current Exon Suggestion module does have limitations, especially for genes with fewer studies or limited internet resources.

### QA Mode.

General-purpose LLMs do not understand advanced biology well. Failure cases have been identified with general-purpose LLMs. The limitations are: (1) information hallucination, (2) lack of up-to-date CRISPR knowledge, (3) absence of peer-reviewed sources, and (4) insufficient problem-solving tailored to user needs. To address these challenges, the QA Mode of CRISPR-GPT involves a multi-source system for answering advanced biology questions. Upon receiving a user request, the QA Mode synthesize information from three sources:

1. A fine-tuned CRISPR-LLama model, using human scientists' discussion threads from a Google Discussion Group, which shows improved problem-solving and troubleshooting capabilities over the baseline model.

2. RAG-based literature retrieval (a Tool Provider agent), which accesses an up-to-date literature database curated by human CRISPR experts, providing peer-reviewed, trustworthy sources for the generated answers. The curated knowledge base includes approximately 50 key publications in the gene-editing field, selected based on citation impact and recency. Using OpenAI Embeddings and FAISS, the system embeds both the database and user queries into semantic vectors, allowing for efficient similarity searches. The top k (k=4) most relevant passages are retrieved based on cosine similarity and ranked by relevance. These retrieved passages are summarized in relation to the user's query and incorporated into the prompt to guide the language model's response generation.

3. General-purpose LLM (for example ChatGPT or LLama).

### Extendibility of CRISPR-GPT.

Given that CRISPR-GPT has a modular multi-agent architecture, integrating new tools and functions into the existing system is easy and training-free. To add a new tool/function, the procedure is as follows:

(1) Tool Wrapping: Develop specific code to encapsulate the tool's functionality within a state machine, which may be referred to as a Tool Provider agent. This wrapper presents user-friendly and LLM-friendly textual interfaces through carefully crafted instructions and responses.

(2) Meta Mode Integration: If one wishes to add the tool to be used in the Meta Mode, the entry state of the new state machine is added to appropriate positions within the relevant predefined workflow.

(3) Auto Mode Integration: Register the entry state of the new tool's state machine in the task decomposition table. This ensures that during task decomposition, the Planner Agent becomes aware of the new tool and can incorporate it into its decision-making process.

### Performance Assessment of CRISPR-GPT.

In this assessment, Gene-Editing-Bench was compiled, which is a collection of test questions and answers for evaluating AI tools' capabilities for CRISPR experimental design, with a total of 288 unique entries covering four topics:

(1) Gene-editing planning: we compiled a total of 50 test cases and answers curated by consensus from human gene-editing experts.

(2) CRISPR guideRNA design: 50 test cases with pre-compiled answers by human experts.

(3) Gene-editing delivery method selection: 50 test cases covering a range of biological systems and major experiment types. For each test case, we asked human experts to rank the available delivery method and report the consensus ranking as answer.

(4) Gene-editing QA: 138 questions and answers, filtered for errors or issues, compiled from both public sources and human experts.

Using this benchmark dataset, individual functions of the CRISPR-GPT agent system were evaluated. Briefly:

(1) Planning evaluation: three batches of subtask lists were generated for each query in the benchmark dataset using CRISPR-GPT. Performance was assessed by comparing these to groundtruth, calculating accuracy, precision, recall, and F1 scores. The task ordering was also evaluated by computing its normalized Levenshtein distance to the groundtruth. For comparison, gpt-4o and gpt-3.5-turbo models were tested. This approach allowed us to assess the LLM Planners' ability to plan and order subtasks for various gene-editing requests.

(2) Delivery method selection evaluation: For each test case, responses were generated using CRISPR-GPT (with and without literature search function), gpt-3.5-turbo, and gpt-4-turbo, letting them propose primary and secondary delivery methods. Responses were evaluated against the ground truth, with the primary method weighted 2 and the secondary method weighted 1. Scores were summed across each request category, allowing assessment of the models' ability to suggest appropriate delivery methods across biological systems.

(3) guideRNA design evaluation: CRISPR-GPT was used to generate gRNA design function lists and parameters, comparing these to the ground truth to calculate accuracy in function selection, order, and parameter specification. For comparison, gpt-4 and gpt-3.5-turbo were also tested with the test set. This approach allowed assessment of the models' ability to interpret user queries and generate appropriate gRNA design strategies.

(4) QA mode evaluation: For evaluation of the QA mode, 40 questions were selected and CRISPR-GPT, gpt-3.5-turbo, and gpt-4 were prompted to generate responses. Three human experts evaluated these responses across four aspects in a full-blind set-up. The experts' scores were averaged to determine each model's final performance, allowing assessment of the models' ability to answer a wide range of gene-editing questions.

To evaluate the real-world applicability of CRISPR-GPT, two independent wet lab demonstrations were conducted:

(1) Beginner Researcher 1: We invited an independent junior PhD scientist, unfamiliar with the CRISPR field, to perform CRISPR gene-editing experiments using CRISPR-GPT via human-agent collaboration. The researcher applied CRISPR-GPT to execute a gene knockout (KO) experiment as part of a cancer research project. The agent provided step-by-step guidance throughout the process. The results were validated through next-generation sequencing and functional assays.

(2) Beginner Researcher 2: An undergraduate student, also unfamiliar with the CRISPR field, was invited to perform gene-editing experiments through collaboration with CRISPR-GPT. The student implemented CRISPR activation in a cancer immunology research project, with stepwise guidance provided by the agent. The results were validated through antibody staining and FACS sorting.

One example of a predefined workflow includes specialized cell-type workflows that may be used to design experiments for specialized cellular applications. To do so, CRISPR-GPT would obtain domain-specific training data on one or more topics (including, e.g., gene-editing efficiency, gene-editing outcomes at target genome, and/or gene-editing protocols or knowledge from prior publications and experimental notes), fine-tune the large language model in the agent using the domain-specific training data, and configure cell type specific task templates. This will enable the platform to design guideRNAs or gene-editing processes in the specialized cell type, and output recommendations accounting for cell-specific parameters and protocols.

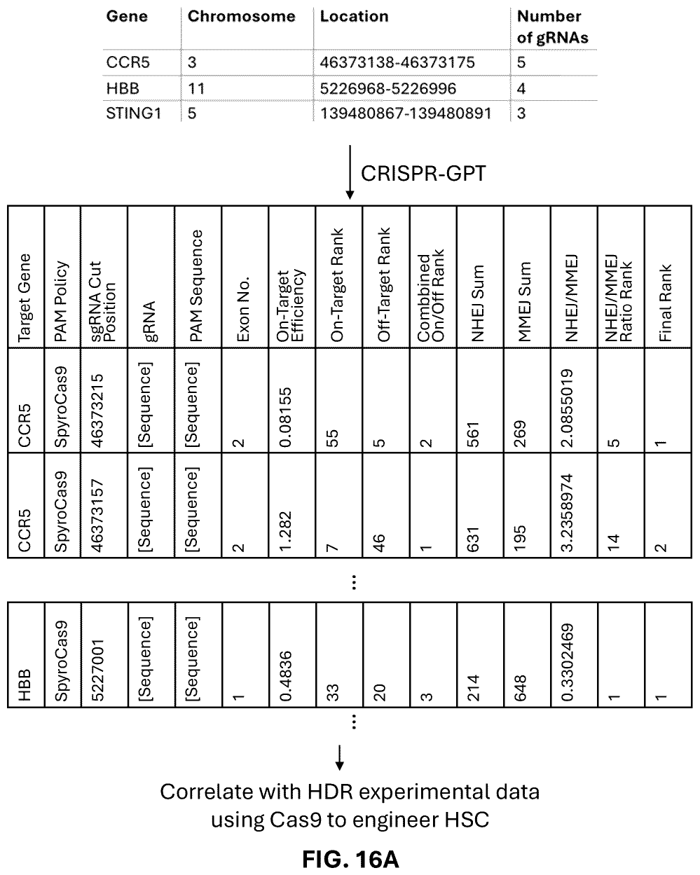

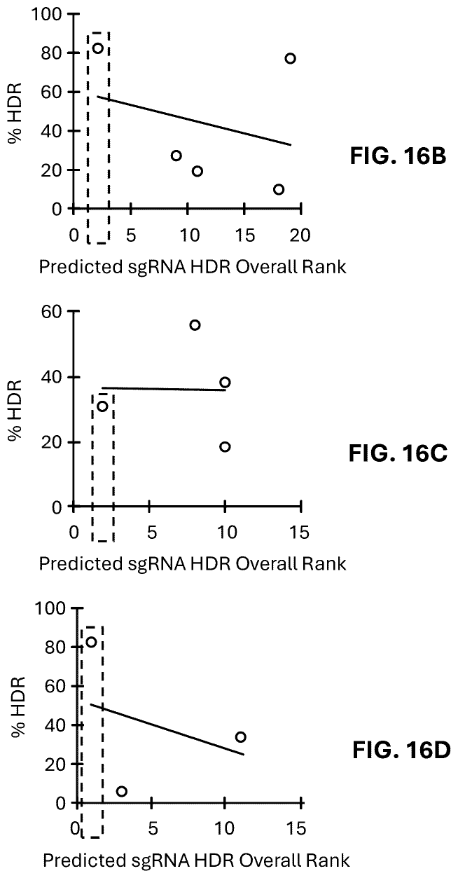

In one example, the concept of specialized application of editing in stem cells, and specifically Hematopoietic Stem Cells (HSC), was considered. CRISPR-GPT may obtain information from previous publications showcasing that: sgRNAs that 1) has high on-target indel, 2) has low off-target, 3) predicted editing outcome favor MMEJ outcomes (defined as deletions greater than or equal to 3 bp) over NHEJ outcomes (<3 bp small indels) have increased rates of HDR-mediate precision editing success. This allows the use of an AI tool that can identify sgRNAs optimized for high editing precision and efficiency based on 1) On-target, 2) Off-target, and 3) predicted MMEJ/NHEJ ratio. See FIG. 16A.

The predicted/identified results can be compared to experimental data. See FIGS. 16B-16D. In FIGS. 16B-16D, for the horizontal axis, rank is from No. 1 (best) on the left with rank increasing as one progresses to the right on the horizontal axis. With rank, a smaller rank number indicates a better predicted efficiency. As such, one would expect an “inverse” relationship between the vertical and horizontal axis if the model is working. As seen, this is indeed overall the case across the three loci shown here (CCR5 (16B), HBB (16C), and Sting1 (16D)), especially CCR5 and Sting1. Further, with the top result highlighted in the dashed line box of each figure, it can be seen that each locus captured a sgRNA with reasonably high HDR (within the top 3 of the generated ranked list).

Thus, it is clear that a specialized CRISPR-GPT tool can be established that other researchers can use to create new reporter cell lines, introduce mutations of interest (e.g. disease modeling or correcting mutation), and/or develop gene therapies. A researcher would simply put in their exact target gene, and what they want to do and then the LLM agent will give them 3-5 guides that ranked highest in terms of the predicted success rate of editing in Hematopoietic Stem Cells (HSC).

A specialized CRISPR-GPT Workflow may include using CRISPR-GPT to locate a target gene and genome region of interest based on location of a given disease mutation (here, HSC mutations). The method may include scanning all CRISPR-Cas9 guideRNA within the region (e.g., ±100 bp). The method may include ranking the guideRNA using CRISPR-GPT to consider on-target (higher better), off-target (lower better), and predicted editing outcome (ratio of MMEJ 3 bp+ deletion vs. <3 bp random-insertion-deletion from NHEJ, higher the better). The method may include outputting the final ranked guideRNA list balancing all factors via LLM reasoning.

Preferably, the models are fine-tuned by the disclosed LLM agent. The models may be fine-tuned using user data or domain-specific data (such as gene-editing efficiency in the case of HSC). The model may be fine-tuned using real-world data from published studies (which may be the source of the domain-specific data). The method may include automatically finding (and preferably ranking) relevant published studies, and importing data from those studies to fine-tune the model.

CRISPR-GPT is a multi-agent, compositional system involving a team of LLM-based agents, including an LLM Planner Agent, a User-Proxy Agent, Task Executor Agents, and Tool Provider Agents. These components are powered by LLMs to interact with one another as well as the human user. The full system may sometimes be referred to as an “agent” to encapsulate the overall functionalities.

To automate biological experiment design, the overall problem may be viewed as sequential decision-making. This perspective frames the interaction between the user and the automated system as a series of decision-making steps, each essential for progressing towards the ultimate goal. Take the Auto Mode for example. A user can initiate the process with a meta-request, for example, “I want to knock out the human TGFBR1 gene in A549 lung cancer cells”. In response, the agent's LLM planner will analyze the user's request, drawing on its extensive internal knowledge base via retrieval techniques. Leveraging the reasoning abilities of the base LLM, the planner generates a chain-of-thought⁴⁴ reasoning path and chooses an optimal action from a set of plausible ones, while following expert-written guidelines. Consequently, the Planner breaks down the user's request into a sequence of discrete tasks, for example “CRISPR/Cas system selection” and “gRNA design for knockout”, while managing inter-dependencies among these tasks. Each individual task is solved by an LLM-powered state machine, via the Task Executor, entailing a sequence of states to progress towards the specific goal. After the meta-task decomposition, the Task Executor will chain the state machines of the corresponding tasks together into a larger state machine and begin the execution process, systematically addressing each task in sequence to ensure the experiment's objectives are met efficiently and effectively.

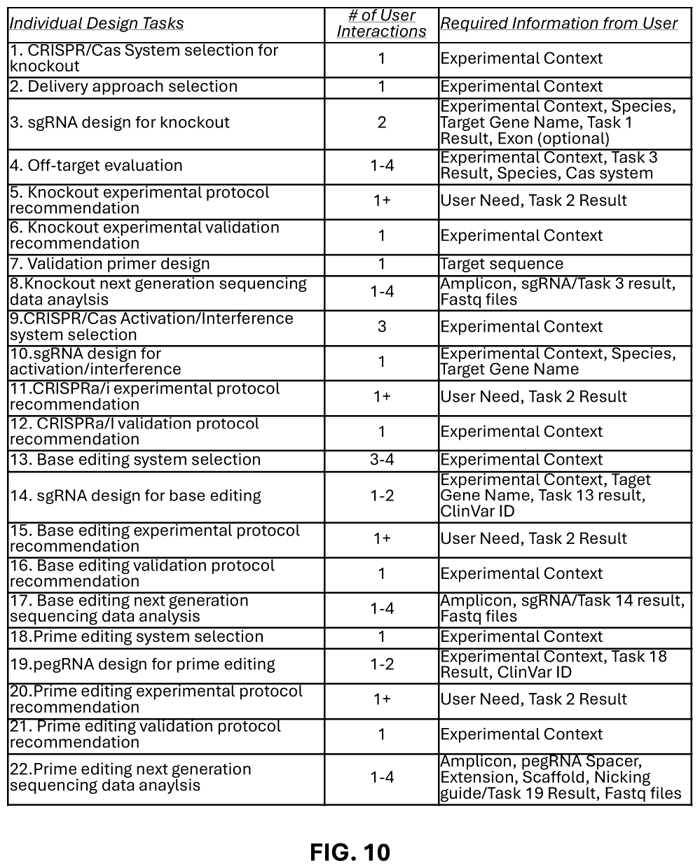

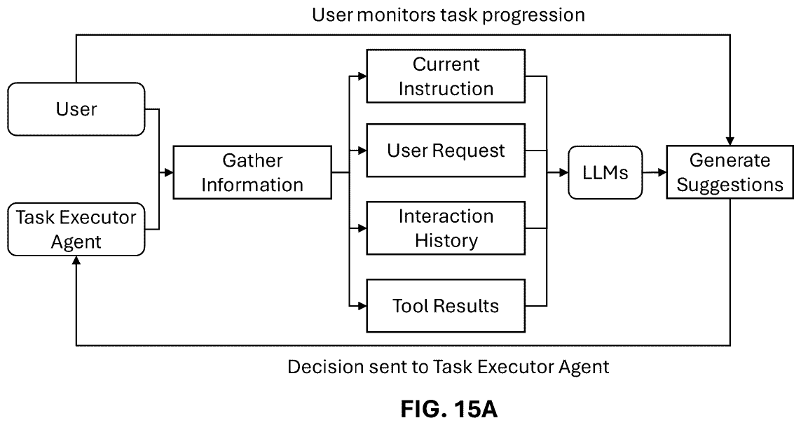

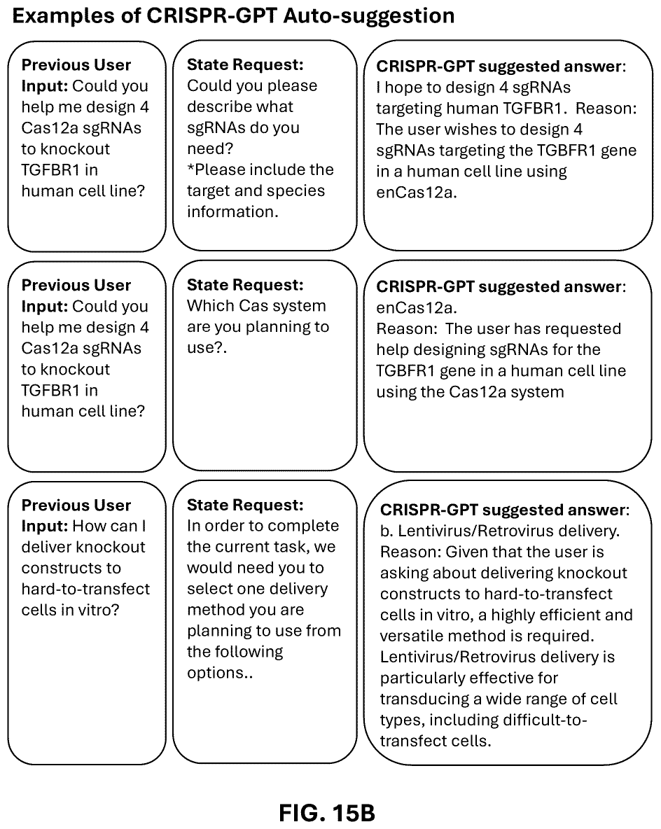

The User-Proxy Agent is responsible for guiding the user throughout the decision-making process via multiple rounds of textual interactions. Typical user interactions required by each task may be seen in, e.g., FIG. 10). At each decision point, the internal state machine presents a “state variable” to the User-proxy Agent, which includes the current task instructions and specifies any necessary input from the user to proceed. The user-proxy agent then interprets this state given the user interactions and makes informed decisions as input to Task Executor on behalf of the user. Subsequently, the User-Proxy Agent receives feedback from the Task Executor, including the task results and the reasoning process that led to those outcomes. Concurrently, the user-proxy agent continues to interact with the user and provides her with instructions, continuously integrating her feedback to ensure alignment with the user's objectives. Sec FIGS. 15A-15B.

To enhance the LLM with domain knowledge, we enable the CRISPR agent to retrieve and synthesize information from published protocols, peer-reviewed research papers, expert-written guidelines, and to utilize external tools and conduct web searches via Tool Provider Agents.

For an end-to-end gene-editing workflow, CRISPR-GPT typically constructs a chain of tasks that includes selecting the appropriate CRISPR system, recommending delivery methods, designing gRNAs, predicting off-target effects, selecting experimental protocols, planning validation assays, and performing data analysis. The system's modular architecture facilitates easy integration of additional functionalities and new tools. CRISPR-GPT serves as a prototype LLM-powered AI co-pilot for scientific research, with potential applications extending beyond gene editing.

CRISPR-GPT is able to automate gene-editing research via several key functionalities. For each functionality the agentic implementation and evaluation results are discussed.

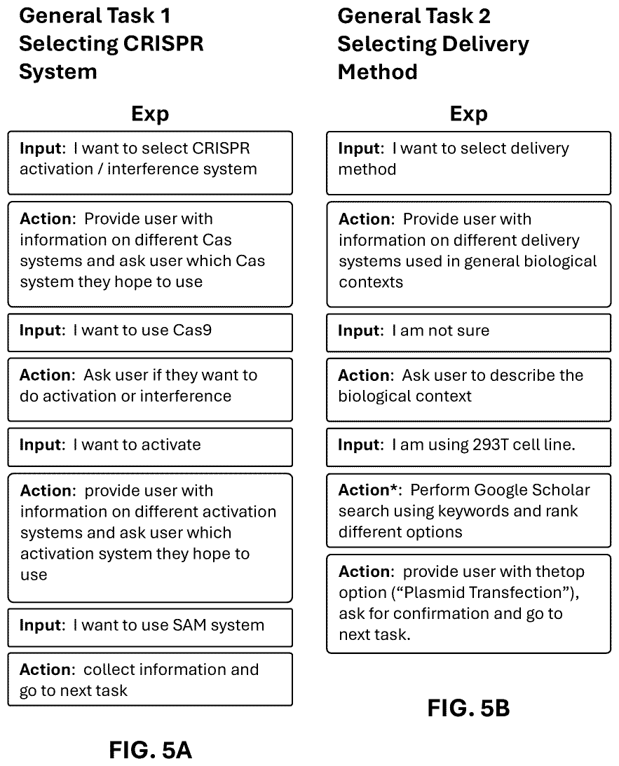

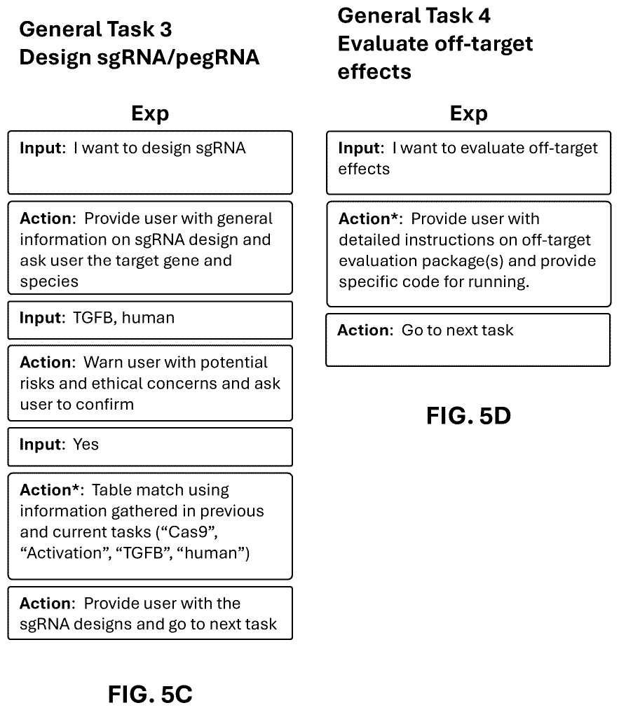

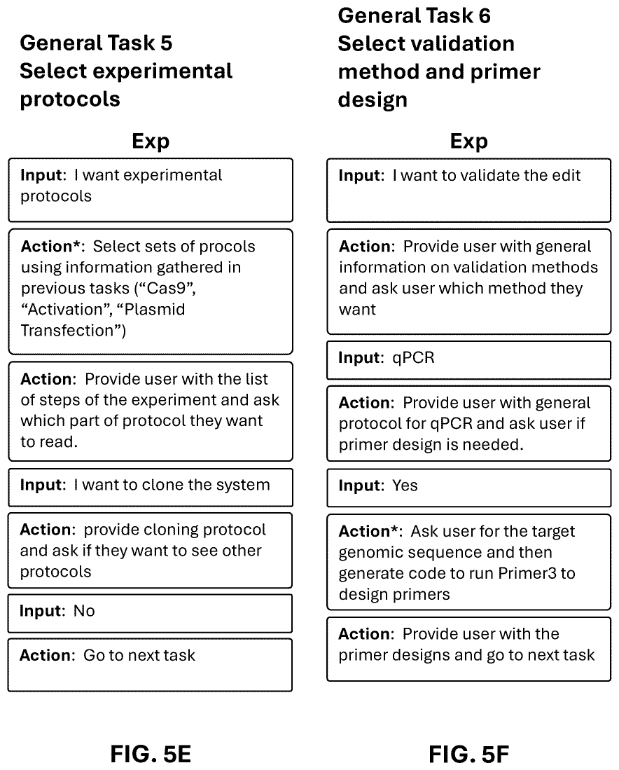

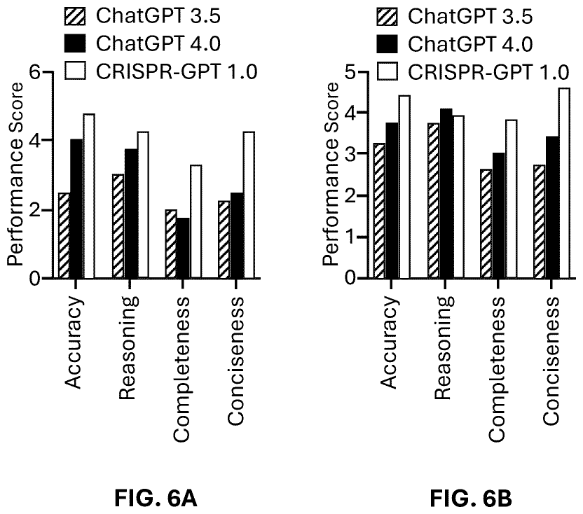

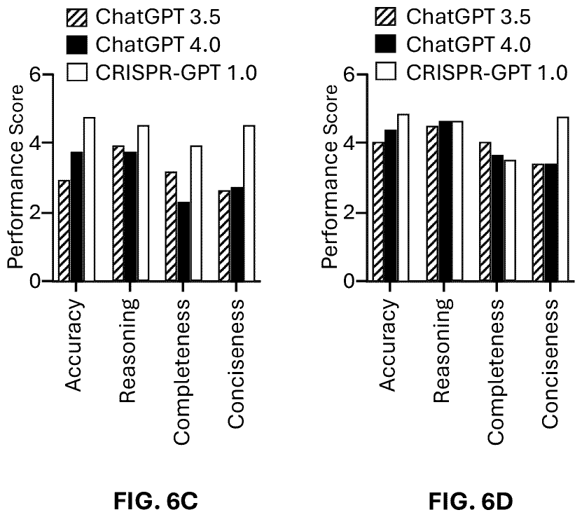

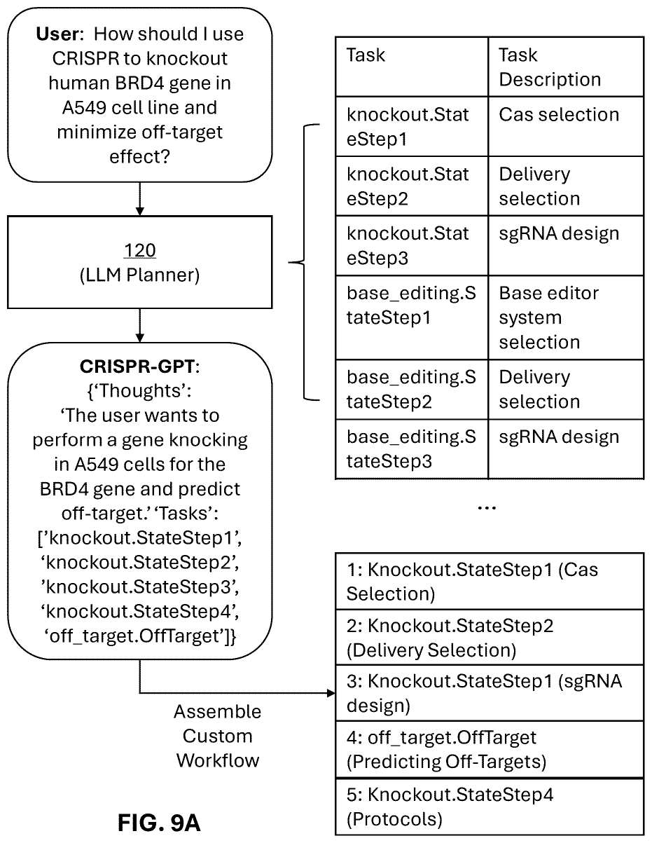

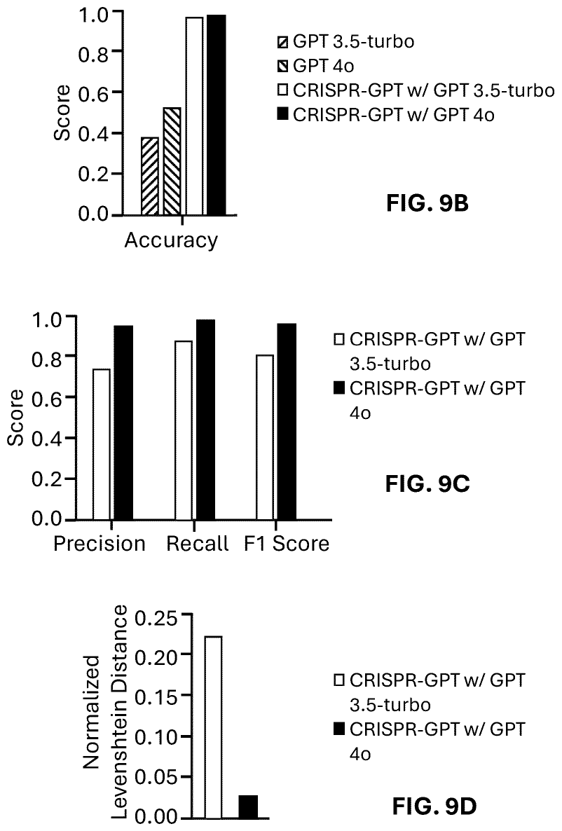

Experiment Planning: The Task Planner Agent is charged with directing the entire workflow and breaking down the user's meta-request into a task chain (see, e.g., Table 1, FIGS. 5A-5F. While the Planner selects and follows a predefined workflow in the Meta Mode, it is able to intake free-style user requests and auto-build a customized workflow in the Auto Mode. For example, a user may only need part of the predesigned workflow including CRISPR/Cas system selection, delivery method selection, guideRNA design and experimental protocol selection before the experiment. Then the Task Planner Agent extracts the right information from the user request and assembles a customized workflow to suit user needs. See FIG. 9A. To evaluate CRISPR-GPT's ability to correctly layout gene-editing tasks and manage inter-task dependence, a planning test set was compiled, as a part of the Gene-editing-Bench, with user requests and golden answers curated by human experts. Using this test set, CRISPR-GPT was evaluated in comparison with prompted general LLMs, showing that CRISPR-GPT outperforms general LLMs in planning gene-editing tasks. The CRISPR-GPT agent driven by GPT-40 scored over 0.99 in accuracy, precision, recall, F1 score, and had less than 0.05 in the normalized Levenshtein distance between agent-generated plans and golden answers. See FIGS. 9B-9D.

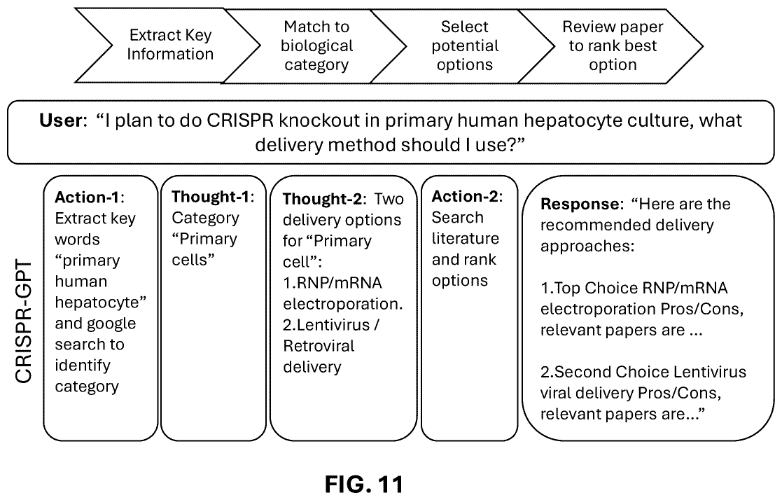

The delivery agent of CRISPR-GPT was presented and evaluated. Delivery is a critical step for all gene-editing experiments. CRISPR-GPT equips LLM with expert-tailored instructions and external tools to choose delivery methods. Specifically, the agent first tries to understand the biological system that the user is planning to edit. It extracts keywords for the target cell/tissues/organisms, performs Google search, and summarizes the results. Then, given its own knowledge and search results, CRISPR-GPT matches the user case with a major biological category—cell lines, primary cells, in vivo, etc.—which reduces the possible options to a focused set of candidate methods. Next, CRISPR-GPT performs literature search with user and method-specific keywords, and ranks the candidate methods based on citation metrics to suggest a primary and a secondary delivery method. See FIG. 11. To evaluate the performance of this module, test cases were compiled, including 50 biological systems as a part of the Gene-editing-Bench. For each case, three human experts were invited to score potential delivery options and utilized those as ground-truth. The output of CRISPR-GPT and baseline models were then evaluated by comparing to the pre-compiled ground-truth score sheet. It was found that CRISPR-GPT outperforms the baseline gpt-4, gpt-3-turbo models. The agent has a substantial edge on difficult tasks such as those involving hard-to-transfect cell lines and primary cell types. It was also noticed that including an additional literature search step improves the agent's performance only moderately.

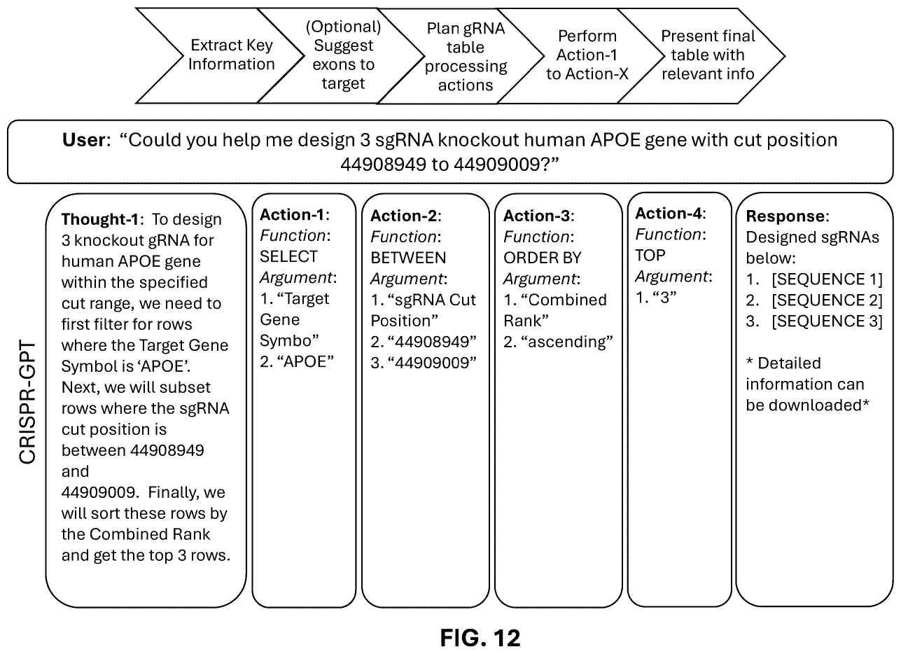

guideRNA design: Good guide RNA (gRNA) design is crucial for the success of CRISPR experiments. Various gRNA design tools and softwares, such as CRISPick and ChopChop, are available. However, it is believed there are two key challenges in general usage: 1. Choosing a trustworthy source. 2. Difficulty in quickly identifying gRNAs that suit specific user requirements or experiment contexts, often requiring lengthy sorting, ranking, or literature review. To address these issues, pre-designed gRNA tables from CRISPick, a reputable and widely used tool, were utilized. The reasoning capabilities of LLMs was leveraged to accurately identify regions of interest, and quickly extract relevant gRNAs. This approach is similar to the recently proposed “chain-of-tables” methodology. See FIG. 12, 14A. To evaluate the ability of CRISPR-GPT to correctly retrieve gRNAs, a gRNA design test set was compiled with ground truth from human experts. CRISPR-GPT agent outperforms the baseline LLMs, in accurately selecting gRNA design actions and configuring the arguments.

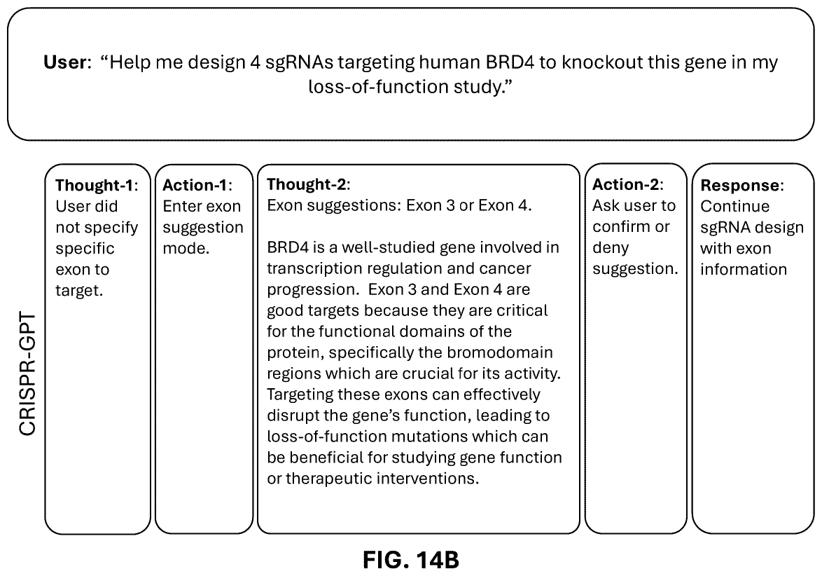

Further, a real-world test case was picked from a cancer biology study, in which many highly-ranked gRNA designs did not generate biological phenotypes, even when their editing efficiencies were high. Instead, the authors of the study had to design gRNAs manually against Exons encoding important functional domains within a gene, and Exon-selected gRNAs induced expected cancer-killing effects. CRISPR-GPT was tested for designing gRNAs targeting BRD4 gene from this study, and the results were compared with those generated by CRISPick and CHOPCHOP. See FIG. 14B. CRISPR-GPT was uniquely able to select the key exons, Exon3-4, within BRD4. In contrast, gRNAs designed by CRISPick or CHOPCHOP would be likely ineffective, as 7 out of 8 gRNAs mapped to non-essential Exons. Taken together, the results support the benefit and validity of this module.

In addition, CRISPR-GPT provides specific suggestions on the choice of the CRISPR system, experimental and validation protocol selection, by leveraging LLM's reasoning ability and retrieving information from an expert-reviewed knowledge base. It also offers automated gRNA off-target prediction, primer design for validation experiments, and data analysis. In particular, the agent provides fully automated solutions to run external softwares, such as Primer3, CRISPRitz and CRISPResso2.

QA Mode with enhanced problem-solving capabilities via fine-tuning LLMs on scientific discussion.

General-purpose LLMs may possess broad knowledge but often lack the deep understanding of science needed to solve research problems. To enhance the CRISPR-GPT agent's capacity in answering advanced research questions, a QA Mode was built that synthesizes information from multiple resources, including published literature, established protocols, and discussions between human scientists, utilizing a combination of RAG technique, a fine-tuned specialized model and a general LLM (e.g., gpt-40).

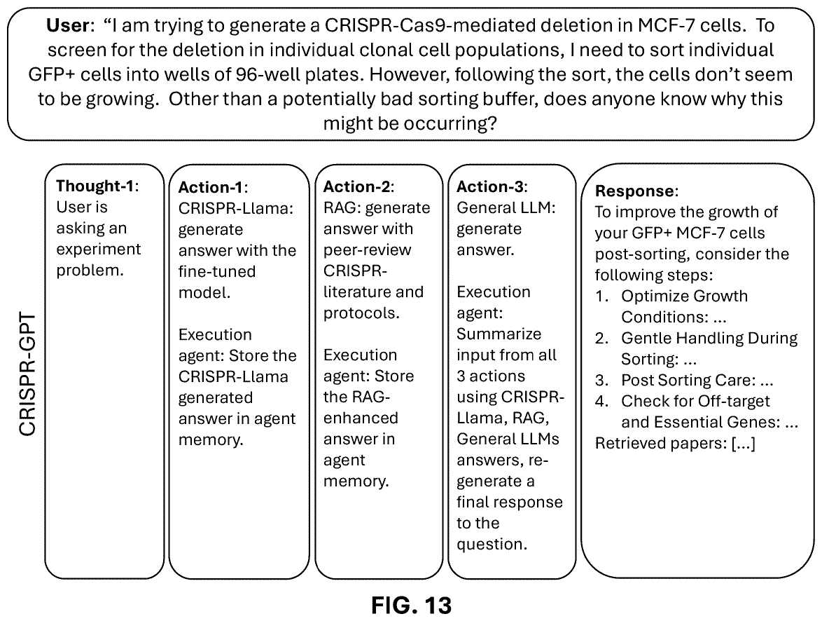

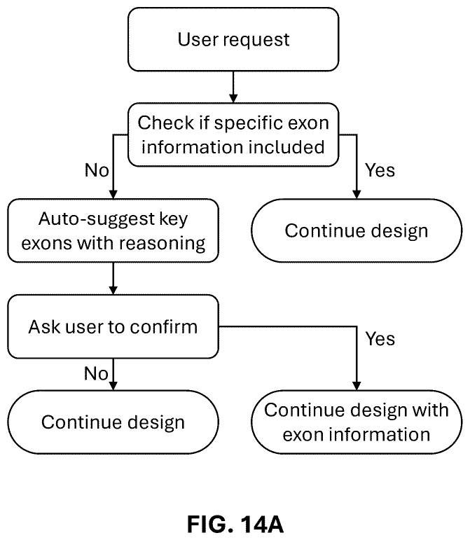

To enhance the QA mode's capacity to “think” like a scientist for problem solving, the inventors sought to train a specialized language model using real scientific discussions among domain experts. The fine-tuned model is used as one of the multiple sources of knowledge for the QA mode. See FIG. 13. To this end, 11 years of open-forum discussions were collected from a public Google Discussion Group on CRISPR gene-editing, starting from 2013. The discussion group involved a diverse cohort of scientists worldwide. This dataset, comprising approximately 4,000 discussion threads, was curated into an instructional dataset with over 3,000 question-and-answer pairs. Using this dataset, an 8-billion-parameter LLM based on the Llama3-instruct model was fine-tuned. The fine-tuned model, which we call CRISPR-Llama3, has improved abilities in gene-editing questions, outperforms the baseline model on basic questions by a moderate 8% and on real-world research questions by ˜20%.

In this example, the Llama3-8B-Instruct model was used, which is an 8-billion-parameter model designed to follow instructions. This model served as the baseline for the fine-tuning experiments. It is capable of general-purpose language understanding but lacks the specific domain expertise required for detailed gene-editing tasks.

More about Llama-8B-Instruct is as follows:

Parameter size directly influences the model's capacity to learn and generalize, with larger models generally having greater flexibility but at the cost of computational requirements. The 8B variant strikes a balance between performance and computational efficiency, making it suitable for use cases where latency and resource constraints are important.

Llama-3B-Instruct is an instruct variant of the Llama models, fine-tuned specifically to follow human instructions. This makes it better at tasks like answering questions, summarizing text, completing tasks based on prompts, and other user-specific instructions. Fine-tuning procedure over Llama-3B-Instruct (which is similar to ChatGPT fine-tuning) helps it align more closely with human expectations and deliver coherent, contextually aware responses to various prompts.

Compared to the pretrained model Llama3-8B, the Llama3-8B-Instruct model has improved abilities in following instructions, reasoning and coding. However, it cannot handle gene-editing tasks well.

The LLama3-8B-Instruct model is open-sourced and is downloaded from the HuggingFace. The training pipeline follows LLama-Factory. LLama-Factory is a unified framework that integrates a suite of cutting-edge efficient training methods and provides a solution for flexibly customizing the fine-tuning of 100+LLMs without the need for coding through the built-in web.

The fine-tuning process involved two following approaches:

(1) Full Parameter Fine-tuning: All model parameters (8 billion) adjusted based on the curated FinalQA dataset. The training precision is float32 (FP32) which occupies 32 bits in computer memory.

(2) QLoRA-based Fine-tuning: QLoRA combines Low-Rank Adaptation, or LoRA, and quantization for the fine-tuning process. LoRA freezes the pre-trained model weights and injects trainable rank decomposition matrices into each layer of the Transformer architecture, reducing the number of trainable parameters. Quantization improves over LoRA by quantizing the transformer model to 4-bit precision. The number of trainable parameters for QLoRA is 3.4 million.

Full Instruction Fine-tuning: Instruction fine-tuning involves training a large language model (LLM) to perform well on tasks where it follows user instructions. It's a process where the model is fine-tuned on labeled datasets, where each input corresponds to a specific desired output. The goal is to align the model's behavior with the human expert's intent. An LLM (e.g. Llama) is typically a neural network based on the transformer architecture⁶⁶. Let the model be parameterized by θ, and given an input question x, the model outputs a probability distribution P_{θ}(y|x) over the possible outputs y. During instruction fine-tuning, the model is trained on pairs (xᵢ, yᵢ), where xᵢ is the question (+context) in FinalQA and yᵢ is the answer. The goal of instruction fine-tuning is to minimize the difference between the model's predicted output and the true output (ground truth) for a given instruction. The standard loss function used is the cross-entropy loss, which measures how well the predicted probability distribution P_{θ}(yᵢ|xᵢ) aligns with the actual distribution. The cross-entropy loss for a single instruction-output pair (xᵢ, yᵢ) is defined as:

$$L ( θ ; {x}_{i} , {y}_{i} ) = - {\sum }_{t = 1}^{T} log {P}_{θ} ( {y}_{i , t} \vert {x}_{i} , {y}_{i , < t} ) ,$$

where y_{i,t} is the token at position t in the output sequence yᵢ, T is the length of the output sequence, P_{θ}(yᵢ,t|x_{i,y}_{_{i<t}}) is the probability assigned by the model to the token y_{i,t}, conditioned on the input xᵢ and all previously generated tokens y_{i<t}. For the FinalQA dataset of instruction-output pairs {(xᵢ, yᵢ)}^{N}ᵢ₌₁, the total loss is:

$$L ( θ ) = \frac{1}{N} {\sum }_{i = 1}^{N} L ( θ ; {x}_{i} , {y}_{i} ) .$$

This loss function encourages the model to assign higher probabilities to correct outputs (i.e., yᵢ) for a given input instruction xᵢ.

To minimize the loss, we update the model parameters using gradient descent. The parameter update rule at step t is given by: θₜ₊₁=θₜ−η∇_{θ}L(θₜ), where the learning rate n is a hyperparameter. In practice, it is common to use AdamW optimizer (a variant of gradient descent) in fine-tuning tasks. It adjusts the learning rate based on past gradients, making it more efficient for training large models.

QLoRA Fine-tuning: LoRA (Low-Rank Adaptation) is a technique for fine-tuning large language models (LLMs) that reduces the number of trainable parameters, making fine-tuning more efficient. Instead of updating all the parameters of the LLM, LoRA introduces low-rank matrices to adapt pre-trained models, considerably reducing the computational and memory overhead. LoRA assumes that weight updates during fine-tuning lie in a low-rank subspace. Instead of directly updating the large weight matrices of the model, LoRA approximates these updates with low-rank matrices. The original large weight matrices are kept frozen, and only the small low-rank matrices are updated.

Let W₀∈R^{d×k} represent a pre-trained weight matrix of the LLM, where d is the input dimension and k is the output dimension. In standard fine-tuning, W₀ would be updated directly, i.e., W=W₀+ΔW, where ΔW is the full-rank weight update matrix learned during fine-tuning. In LoRA fine-tuning, instead of learning the full-rank matrix ΔW, one could decompose it into two low-rank matrices: ΔW=AB^{T}, where: A∈R^{d×r}, B∈R^{r×k}, where r is much smaller compared to d or k. This means that during fine-tuning, the matrices A and B are being learned, both of which have much fewer parameters compared to W₀. The updated weight matrix becomes: W=W₀+AB^{T}.

Loss Function. For LoRA fine-tuning, it also uses the cross-entropy loss. For an input x and its corresponding label y, the trainable parameters are low-rank matrices A, B with the loss

$$L ( A , B ; x , y ) = - {\sum }_{t = 1}^{T} log {P}_{θ} ( {y}_{t} \vert x , {y}_{< t} ) .$$

The difference is that instead of optimizing the full W, we are now optimizing the low-rank matrices A and B.

Gradient update. The goal is to minimize the loss with respect to A and B. The gradient update for LoRA also follows the gradient descent mechanism (with n being the learning rate):

$${A}_{t + 1} = {A}_{t} - η {∇}_{A} L ( {A}_{t} , {B}_{t} ; x , y ) ; {B}_{t + 1} = {B}_{t} - η {∇}_{B} L ( {A}_{t} , {B}_{t} ; x , y ) .$$

Since A and B are much smaller than W₀, the computational cost is considerably reduced.

Quantization. QLoRA extends LoRA by applying quantization to the frozen pre-trained weights in order to further reduce memory usage. The model weights are quantized into lower-precision formats (e.g., 4-bit), which allows for loading much larger models into memory. At the same time, QLoRA retains LoRA's low-rank adaptation for efficient fine-tuning.

Training command: For QLora training, the following command was applied:
CUDA_VISIBLE_DEVICES-0 llamafactory-cli train examples/lora_single_gpu/llama3_lora_sft.yaml.

For Full training, the following command was applied:
CUDA_VISIBLE_DEVICES=0,1,2,3 python-m torch.distributed.run \--nproc_per_node $NPROC_PER_NODE--nnodes 1--standalone \src/train.py full_fine_tunning/single_node.yaml

In the commands, CUDA_VISIBLE_DEVICES specifies how many GPUs to use within a compute node, and the python and yaml files can be found in LLama-Factory Github.

Detailed parameters and configurations used are:

| | Full | OLoRA |
|---|---|---|
| Hyper-parameters | Fine-Tuning | Fine-Tuning |
| Learning Rate | 5e−6 | 1e−4 |
| Fine-tuning Type | Full | Lora |
| Quantization Bit | NA | 4 bits |
| Per_device_train_batch_size | 16 | 16 |
| Gradient_accumulation_steps | 8 | 8 |
| Training_epochs | 6 | 15 |
| Lr_scheduler_type | cosine | cosine |
| Warmup_steps | 0.05 | 0.05 |
| Distributed Training | Deepspeed | NA |
| | | (Trained on a |
| | | single GPU) |
| Optimizer | adamw_torch | adamw_torch |
| Dataset | FinalQA | FinalQA |

Choice of epoch number: During training, the number of training epochs was varied, and it was found that finetuning >15 epochs does not help. In particular, full-parameter fine-tuning for 20 epochs did not improve the performance in gene-editing questions compared to CRISPR-Llama3 trained with 6 epochs. This was tested for multiple-choice questions in the FinalQA dataset, and it attained a score of 90% that was only comparable to CRISPR-Llama3 (91%). What's more important is that fine-tuning for >15 epochs actually degrades the model's performance on general questions, due to over-optimization/overfitting to the small dataset used for finetuning.

The choice of epoch number and observation of overfitting is consistent with common practice in LLM research. In general LLM research, while the base model is often pretrained on large amount of data entries using a large number of epochs, finetuning usually takes only a few epochs (2-15 epochs). The reason is that finetuning a model on a small domain specialized dataset could easily cause overfitting and catastrophic forgetting. A model that is “over-finetuned” could appear to memorize the dataset used for finetuning, but it cannot generalize the knowledge and even forget common senses learnt via pre-training. The LIMA paper suggests that supervised fine-tuning (SFT) only requires a small demonstration dataset. In their setting, they finetuned llama with 15 epochs with 1000 curated (question, response) pairs and showed remarkable performances. The BERT paper finetuned its model using only 2-3 epochs, and the ROBERTa paper finetuned its model using 10 epochs.

This fine-tuned LLM was integrated into the QA Mode as a “brainstorming source”, enabling the agent to generate ideas like a human scientist and provide a second opinion for difficult queries.

To assess the performance of the QA Mode, the Gene-editing-Bench QA test set was used. The test questions encompass basic gene-editing knowledge, experimental troubleshooting, CRISPR application in various biological systems, ethics and safety. CRISPR-GPT, gpt-3.5-turbo, and gpt-40 were prompted to generate responses to test questions. Three human experts scored the answers in a fully blinded setting. The test demonstrated that the QA Mode outperformed baseline LLMs in accuracy, reasoning, and conciseness, with improvement of 12%, 15%, and 32%, respectively, versus GPT-40. Human evaluators observe that general-purpose LLMs sometimes make factual errors and tend to provide extensive answers not all relevant to the questions. For example, one question is about solving cell growth issues in an experiment where a scientist performed Cas9 editing followed by single-cell sorting using MCF-7 cells. For this question, the QA Mode provided a concise, accurate summary of potential reasons and actionable solutions. In contrast, GPT-40 responded with a long list of 9 factors/options, but at least 2 of them were not applicable to MCF-7 cells. This, and other examples showcase the advantage of CRISPR-GPT QA Mode. Overall, evaluation results confirmed that the multi-source QA Mode is significantly better at answering advanced research questions about gene-editing.

To further evaluate the human user experience of CRISPR-GPT, we assembled a panel of eight gene-editing experts to assess the agents' performance for end-to-end experiment designs covering all 22 individual tasks. The experts were asked to rate their experiences in four dimensions: Accuracy, Reasoning and Action, Completeness, and Conciseness. CRISPR-GPT demonstrated improved accuracy and strong capabilities in reasoning and action, whereas general LLMs, such as GPT-40, often included errors and were prone to hallucination.

Highlighted by human evaluators' observations, the CRISPR-GPT agent provides users with more accurate, concise, and well-rounded instructions to execute the planned experiments. The ability of CRISPR-GPT to perform specialty gene-editing tasks, such as exon-selected gRNA design, customized off-target prediction, and automated sequencing data analysis, reinforced its advantage versus general-purpose LLMs. This is confirmed by the task-specific evaluation results. Despite its strengths, CRISPR-GPT struggled with highly complex requests and rare biological cases, highlighting areas for improvement.

In the real-world wet-lab experiments discussed herein, in the first test, the junior researcher conducted gene knockouts in the human A549 lung adenocarcinoma cell line, targeting four genes involved in tumor survival and metastasis: TGFBR1, SNAI1, BAX, and BCL2L1. The experiment was designed from scratch with CRISPR-GPT. Based on user-AI interaction, enAsCas12a was selected for its multi-target editing capability and low off-target effects. For delivery, CRISPR-GPT recommended lentiviral transduction for stable Cas and gRNA expression. The gRNAs for the four target genes were designed through CRISPR-GPT. Furthermore, CRISPR-GPT provided step-by-step protocols for gRNA cloning, lentivirus production, and viral delivery into A549 cells. To validate the editing, the researcher followed CRISPR-GPT's NGS protocol, using assay primers designed via the integrated Primer3 tool. After generating the NGS data, the raw sequencing files were uploaded into CRISPR-GPT for automated analysis through the CRISPResso2 pipeline. The analysis reports, sent directly via email, summarized the editing outcomes and showed consistently ˜80% high efficiency across all target genes. To further assess the biological phenotypes of TGFBR1, SNAIL knockout in A549 cells, the researcher conducted an Epithelial-mesenchymal transition (EMT) induction experiment by treating A549 cells with TGFβ. The qPCR results revealed that the knockout A549 cell lines (A549 TGFBR1 KO and A549 SNAI1 KO) showed up to 9-fold reduction in CDH1 expression change, and up to 34-fold reduction in VIM expression change, which are both key marker genes in the EMT process. This confirms the biological role of TGFBR1 and SNAI1 signaling in driving EMT progression (a crucial driver of metastasis) in lung cancer cells.

In the second experiment, the junior researcher performed epigenetic editing to activate two genes involved in cancer immunotherapy resistance in a human melanoma model cell line. CRISPR-GPT guides the researcher through the full workflow: identify the most suitable CRISPR activation system, select an appropriate delivery method for A375 cells, design dCas9 gRNAs (three gRNAs per gene), and generate protocols for validating editing outcomes. After editing was completed, measurements of target protein expression level confirmed successful activation of both genes, with up to 56.5% efficiency for NCR3LG1, and 90.2% efficiency for CEACAM1, when comparing gRNA-edited groups vs. negative control gRNAs.

A number of implementations have been described. Nevertheless, it will be understood that various modifications may be made without departing from the spirit and scope of the disclosure. Accordingly, other implementations are within the scope of the following claims.

1. A platform for automated design of gene-editing experiments, comprising:
one or more processing units; and
a non-transitory computer-readable storage device operably coupled to the one or more processing units, the non-transitory computer-readable storage device containing instructions that, when executed, configures the one or more processing units to, collectively, perform a method, the method comprising:
receiving a meta request, the meta request including information about a requested gene-editing experiment;
configuring, via a reasoning framework, an ordered list of tasks required to achieve the requested gene-editing experiment based on the information, the reasoning framework configured to sequentially send each task in the ordered list of tasks, and optionally a previous result, to a Task Executor module and receive a result from the Task Executor Module responsive to the task;
implementing, via the Task Executor module, a task received from the reasoning framework, the Task Executor utilizing state machines to decompose sub-goals, the Task Executor configured to:
connect to one or more external application programming interfaces (APIs) by sending an API call to a Tool Provider module and receiving a result;
provide instructions to a User-Proxy Agent module and receive user input responsive to the instructions from the User-Proxy Agent, and send feedback to the User-Proxy Agent based on the task and/or user input; and
forming a prompt, via the User-Proxy Agent module, based on an instruction inherent to a current state from the Task Executor module, a request made by the user, a history of past interactions within the current task session, results from external APIs, or a combination thereof, then using the prompt with the User-Proxy Agent module to determine an appropriate next action, the current state encapsulating a description of a current task and any input required from a user;
where the platform is configured to output one or more recommendations responsive to the meta request.

2. The platform of claim 1, wherein the reasoning framework comprises a large language model configured to decompose the meta request into the ordered list of tasks.

3. The platform of claim 2, wherein the large language model is trained using a dataset comprising curated question-and-answer pairs derived from gene-editing discussions.

4. The platform of claim 3, wherein the large language model is fine-tuned using a technique selected from the group consisting of full parameter fine-tuning and quantized low-rank adaptation (QLoRA) fine-tuning.

5. A method for selecting gene editing delivery methods, comprising:
extracting parameters from user inputs related to gene editing;
performing a literature search based on the extracted parameters;
ranking candidate delivery methods using citations from the literature search results; and
outputting a ranked list of candidate delivery methods.

6. The method of claim 5, further comprising categorizing the user inputs into one of a plurality of predefined biological categories, wherein the literature search is performed based on the categorized biological category.

7. The method of claim 6, wherein the plurality of predefined biological categories comprises: mammalian in vivo, mammalian embryos, mammalian primary cells or stem cells ex vivo, mammalian cell lines with strong evidence of high-efficiency transfection, mammalian cell lines or organoids without strong evidence of high-efficiency transfection, human in vivo or human embryos, and bacteria, viruses, and other organisms.

8. The method of claim 7, wherein ranking the candidate delivery methods comprises:
retrieving a predefined set of delivery methods associated with the categorized biological category;
calculating a score for each delivery method based on the number of citations from the literature search results; and
ordering the delivery methods based on the calculated scores.

9. A method for training a gene editing model, comprising:
obtaining a dataset of gene editing discussions from a public forum;
preprocessing the dataset to extract question-answer pairs;
fine-tuning a pre-trained language model using the extracted question-answer pairs; and
storing the fine-tuned model for subsequent use in gene editing tasks.

10. The method of claim 9, wherein preprocessing the dataset comprises:
anonymizing personal information in the discussions;
extracting question-answer pairs from individual discussion threads; and
filtering the extracted pairs to remove irrelevant or low-quality content.

11. The method of claim 10, wherein fine-tuning the pre-trained language model comprises using a technique selected from the group consisting of full parameter fine-tuning and quantized low-rank adaptation (QLoRA) fine-tuning.

12. The method of claim 11, further comprising:
evaluating the fine-tuned model using a test set of gene editing questions; and
comparing the performance of the fine-tuned model to the pre-trained model on the test set.

13. A method for gene editing inference, comprising:
receiving a gene editing query;
processing the query using a model trained with fine-tuning on gene editing discussions;
retrieving relevant information from a curated knowledge base of gene editing literature;
synthesizing an answer based on the processed query and retrieved information; and
outputting the synthesized answer.

14. The method of claim 13, wherein retrieving relevant information from the curated knowledge base comprises:
embedding the gene editing query and documents in the knowledge base into semantic vectors;
performing a similarity search to identify the most relevant documents based on cosine similarity between the query vector and document vectors; and
summarizing the identified relevant documents in relation to the gene editing query.

15. The method of claim 14, wherein synthesizing the answer comprises:
combining information from the processed query, the retrieved relevant information, and a response generated by a fine-tuned large language model trained on gene editing discussions; and
generating a concise answer that addresses the specific aspects of the gene editing query.

16. A method for designing guide RNA for gene editing, comprising:
receiving a user request for guide RNA design;
extracting relevant parameters from the user request;
accessing a pre-designed guide RNA table;
applying a chain-of-table methodology to process the pre-designed guide RNA table based on the extracted parameters;
selecting guide RNA sequences from the processed table; and
outputting the selected guide RNA sequences.

17. The method of claim 16, wherein applying the chain-of-table methodology comprises:
selecting rows from the pre-designed guide RNA table where specified columns match given values;
ordering the selected rows based on values in a specified column; and
returning a top number of rows from the ordered selection.

18. A system for automated design of gene-editing experiments, comprising:
a User-Proxy Agent module configured to interact with a user and process user inputs;
a reasoning framework configured to decompose a gene editing request into an ordered list of tasks;
a Task Executor module configured to implement tasks using state machines;
a Tool Provider module configured to connect to external APIs; and
a non-transitory computer-readable storage device containing instructions that, when executed, cause the system to perform a method comprising:
receiving a gene editing request from the user via the User-Proxy Agent module;
decomposing the request into tasks using the reasoning framework;
sequentially executing the tasks using the Task Executor module; and
outputting gene editing experiment design recommendations to the user via the User-Proxy Agent module.

19. The system of claim 18, wherein the reasoning framework comprises a large language model trained on a dataset of curated question-answer pairs derived from gene-editing discussions.

20. The system of claim 19, wherein the large language model is fine-tuned using a technique selected from the group consisting of full parameter fine-tuning and quantized low-rank adaptation (QLoRA) fine-tuning.
